(19) United States
(12) Patent Application Publication
(10) Pub. No.: US 2024/0293482 A1
(43) Pub. Date: Sep. 5, 2024

(54) COMPOSITIONS AND METHODS FOR TREATING BIOFILMS, INFECTIONS AND PERIODONTITIS

(71) Applicant: Etheim Biotics, LLC, Gaithersburg, MD (US)

(72) Inventors: Karen E. NELSON, Sandy Spring, MD (US); Manny TORRALBA, Sandy Spring, MD (US); Ahmed MOUSTAFA, Cairo (EG); Josue HERNANDEZ, Gaithersburg, MD (US); L. Anthony HILL, Sandy Spring, MD (US)

(21) Appl. No.: 17/796,439

(22) PCT Filed: Jan. 28, 2021
(86) PCT No.: PCT/US2021/015434

§ 371 Date: Jul. 29, 2022

Related U.S. Application Data
(60) Provisional application No. 62/967,454, filed on Jan. 29, 2020.

(51) Int. Cl. A61K 35/744; A01N 63/20; A01P 1/00; A61L 2/00; A61L 2/18; A61P 1/02

(52) U.S. Cl. CPC A61K 35/744; A01N 63/20; A01P 1/00; A61L 2/0088; A61L 2/18; A61P 1/02; A61L 2202/21; A61L 2202/24

(57) ABSTRACT

In alternative embodiments, provided are compositions, including products of manufacture, pharmaceutical compositions and kits, and methods, for treating, ameliorating, preventing or reducing the growth of, a biofilm, such as a biofilm in an oral environment, such as a biofilm growing on or adherent to a tooth an implant, or an oral prosthetic, or microbial colonies found in biofilms. In alternative embodiments, provided are compositions, including products of manufacture and kits, and methods, for treating, ameliorating, preventing or reducing the severity of an infection. In alternative embodiments, antibacterial and therapeutic formulations as provided and used herein are derived or isolated from Streptococcus sanguinis and/or Staphylococcus epidermidis organisms or cultures.

### RELATED APPLICATIONS

This Patent Convention Treaty (PCT) International Application claims the benefit of priority to U.S. Provisional Application Ser. No. (USSN) 62/967,454, Jan. 29, 2020. The aforementioned application is expressly incorporated herein by reference in its entirety and for all purposes.

### TECHNICAL FIELD

This invention generally relates to microbiology and dentistry. In alternative embodiments, provided are compositions, including products of manufacture, pharmaceutical compositions and kits, and methods, for treating, ameliorating, preventing or reducing the growth of, a biofilm, such as a biofilm in an oral environment, such as a biofilm growing on or adherent to a tooth, an implant, or an oral prosthetic, or microbial colonies found in biofilms. In alternative embodiments, provided are compositions, including products of manufacture and kits, and methods, for treating, ameliorating, preventing or reducing the severity of an infection. In alternative embodiments, antibacterial and therapeutic formulations as provided and used herein are derived or isolated from Streptococcus sanguinis and/or Staphylococcus epidermidis organisms or cultures.

### BACKGROUND

Periodontal disease is characterized by a chronic inflammatory host response and the lack of normal resolution. The immune response system reacts to pathogens in the biofilm in an attempt to eliminate the bacteria. A chronic inflammatory state may allow the persistence of periodontal pathogens within the oral biofilm if not resolved.

Many studies have reported comorbidities between periodontitis and other disease and conditions. These diseases and conditions include various cancers, cardiovascular disease, pulmonary disease, dementia, kidney disease, rheumatoid arthritis, Type-2 diabetes (T2D), lupus and smoking, among others. Periodontal bacteria and periodontal bacterial DNA have been located at sites distant from the oral cavity, including for example serum and synovial fluid in rheumatoid arthritis (RA) patients and synovial fluid in RA patients, where Porphyromonas gingivalis has been found. Additionally, correlations between dysbiosis in the oral microbiome and systemic diseases have been shown for Alzheimer's disease, preterm birth (see e.g., Cobb et al. 2017), cardiovascular diseases, and colorectal cancers. Center for Disease Control (CDC) data reveals that smokers have a rate of periodontitis approximately 5 times higher than that of nonsmokers (see CDC: QuickStats: Percentage of Adults Aged 20-64 Years with Periodontitis, by Smoking Status and Sex.).

Human oral biofilms are complex multi-dimensional structures that form on stratum, including the oral cavity. When a quorum of bacteria develops, they organize themselves into structures that are connected by channels that enable access to food and the elimination of end products (waste). This matrix of extracellular polymeric substances (EPS) while allowing for passage of different molecules also allows the cells to develop a protective mechanism, which may include the development of resistance to antibiotics. This matrix is recalcitrant to antimicrobial therapy, and is difficult to eradicate. Studies have shown that bacteria within a biofilm matrix are up to 1000 times more resistant to antibiotics than in a planktonic state. Additionally, the immune response of the host may be ineffective against bacteria when they are present in a biofilm versus a planktonic state; Pseudomonas aeruginosa and Porphyromonas gingivalis, among others, exhibit this characteristic. Accordingly, better treatments for periodontitis are needed.

### SUMMARY

In alternative embodiments, provided are products of manufacture, or pharmaceutical compositions or formulations comprising:
(a)
(1) a Streptococcus sanguinis bacterial strain,
wherein optionally the Streptococcus sanguinis bacterial strain:
   (i) comprises or has contained therein a 16S IRNA gene sequence with: at least 96%, 97%, 98%, 99% or 99.5% or more, or complete (100%) percent sequence identity to the sequence of SEQ ID NO:1 (a strain having complete (100%) percent sequence identity to SEQ ID NO:1 is also called EGEN14),
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62,
   (ii) is a strain having ATCC deposit no. [ ], or
   (iii) comprises or has contained therein a 16S rRNA gene sequence having at least 96%, 97%, 98%, 99% or 99.5% or more, or complete (100%) percent sequence identity to a16S rRNA gene of a Streptococcus sanguinis SK36 strain;
(2) a Staphylococcus epidermidis bacterial strain,
wherein optionally the Staphylococcus epidermidis bacterial strain:
   (i) comprises or has contained therein a 16S rRNA gene sequence with: at least 96%, 97%, 98%, 99% or 99.5% or more, or complete (100%) percent sequence identity to the sequence of SEQ ID NO:2 (a strain having complete (100%) percent sequence identity to SEQ ID NO:2 is also called EGEN68),
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62,
   (ii) is a strain having ATCC deposit no. 12228, or
   (iii) comprises or has contained therein a 16S rRNA gene sequence having at least 96%, 97%, 98%, 99% or 99.5% or more, or complete (100%) percent sequence identity to a16S rRNA gene of a Staphylococcus epidermidis strain ATCC deposit no. 12228,
wherein the Streptococcus sanguinis and/or the Staphylococcus epidermidis in the product of manufacture, pharmaceutical composition or a formulation are substantially live and viable and are capable of secreting a bacteriocin capable of inhibiting or slowing the growth of a biofilm-forming bacterium or inhibiting or slowing the formation of a biofilm; or
(3) a combination or mixture of bacterial strain comprising at least one bacterial strain from (1) and at least one bacterial strain from (2);
(b) a supernatant or culture medium of a culture or fermentation of the Streptococcus sanguinis and/or Staphylococcus epidermidis bacterial strain, wherein the supernatant or culture medium comprises a bacteriocin capable of inhibiting or slowing the growth of a biofilm-forming bacterium or inhibiting or slowing the formation of a biofilm,
and optionally the supernatant or culture medium is free or substantially free of any bacteria, and optionally a culture or a fermentation supernatant or culture medium is filtered to remove substantially all or all bacteria from the supernatant or culture medium;
(c) a fraction or isolate of the supernatant or culture medium of (b), wherein the fraction or isolate comprises a bacteriocin capable of inhibiting or slowing the growth of a biofilm-forming bacterium or inhibiting or slowing the formation of a biofilm,
wherein optionally the fraction or isolate is prepared by a column chromatography of the supernatant or culture medium, and the fraction or isolate comprises an eluate fraction of the column capable of inhibiting or slowing the growth of a biofilm-forming bacterium or inhibiting or slowing the formation of a biofilm; or
(d) the product of manufacture, pharmaceutical composition or a formulation of (a), (b) or (c), further comprising a pharmaceutically acceptable excipient, diluent, or carrier,
wherein optionally the biofilm-forming bacterium is a genus or a species of Actinomycetes, Bacillus, Listeria (optionally, a L. monocytogenes), Staphylococcus, Escherichia (optionally, a E. coli), Pseudomonas (optionally, a P. aeruginosa), Corynebacterium, Haemophilus, Aggregribacter, Porphyromonas, Neisseria, Capnocytophaga, Fusobacterium and/or Leptotrichia, and/or a lactic acid bacteria (LAB) (optionally, a Bifidobacterium, Lactobacillus, Lactococcus (optionally, a L. lactis), Leuconostoc, Pediococcus, Streptococcus (optionally, S. sanguinus), Aerococcus, Alloiococcus, Carnobacterium, Dolosigranulum, Enterococcus, Oenococcus, Tetragenococcus, Vagococcus or Weissella.

In alternative embodiments of pharmaceutical compositions or formulations as provided herein:
the Streptococcus sanguinis and/or Staphylococcus epidermidis bacterial strain, or bacterial strain as provided herein, is lyophilized or freeze-dried, and optionally a unit dosage comprises lyophilized or freeze-dried substantially viable Streptococcus sanguinis and/or Staphylococcus epidermidis;
the Streptococcus sanguinis and/or Staphylococcus epidermidis bacterial strain, or a bacterial strain as provided herein, is present in an amount that comprises from between about 1×10² to about 1×10¹², or 1×10³ to about 1×10¹¹ CFU/gram (g) of the bacterial strain with respect to a total weight of the pharmaceutical composition, or is present in an amount that comprises from about 1×10² to about 1×10¹², or 1×10³ to about 1×10¹¹ CFU per unit dose;
the pharmaceutical composition or formulation is formulated as or included or contained in: a liquid, a gel, a capsule, a pill, a tablet, a geltab, a lozenge, a powder, a gum, a sachet, a cachet, an elixir, a hydrogel or a viscosity enhancing agent, a suspension, an emulsion, a liposome or a lipid carrier, a hydrogel, a solution, a toothpaste, a mouthwash, a syrup, a food, a chewing gum, a paste, a candy, a confectionary, an aerosol, a lysosome, a microparticle, a nanoparticle, a microsphere, or a lyophilate or an equivalent thereof; and/or
the pharmaceutical composition or formulation further comprises: an additional bacteriocin or biofilm disrupting agent; or, a phage, a stabilizer, an enzyme, a surfactant (optionally a biosurfactant), a hydrogel or viscosity enhancing agent, an antibiotic or a antimicrobial, a complexing agent, a buffer, an emulsifier, a natural product, peroxide, a flavoring agent, a preservative, a tissue (e.g., gingival or periodontal tissue) penetration enhancer, a pharmaceutically acceptable rate modifying agent, a sustained-release polymer, anti-inflammatory agents, immune-suppressive agents, immune-stimulatory agents, dentinal desensitizers, an odor masking agent, or any combination thereof.

In alternative embodiments, provided are methods for treating, ameliorating, preventing or reducing the growth of: a biofilm, optionally a biofilm in an oral environment, optionally a biofilm growing on or adherent to a tooth, a medical device (optionally a bone implant, a pin, a mesh, a stent or an artificial valve), an implant or a prosthetic (optionally an ocular lens), optionally an oral implant or oral prosthetic; or, a microbial colony found in a biofilm, comprising: administering in an individual in need thereof, or applying to a surface in need thereof, a pharmaceutical composition or formulation as provided herein.

In alternative embodiments, provided are methods for treating, ameliorating, preventing or reducing the growth of, a biofilm-associated microorganism, or converting the biofilm-associated microorganism to a planktonic state, comprising: administering in an individual in need thereof, or applying to a surface in need thereof, a pharmaceutical composition or formulation as provided herein,
wherein optionally the biofilm-associated microorganism comprises a bacterium from a genus or a species of: Actinomycetes, Bacillus, Listeria (optionally L. monocytogenes), Staphylococcus, Escherichia (optionally E. coli), Pseudomonas (optionally P. aeruginosa), Corynebacterium, Haemophilus, Aggregribacter, Porphyromonas, Neisseria, Capnocytophaga, Fusobacterium and/or Leptotrichia, and/or a lactic acid bacteria (LAB) (optionally a Bifidobacterium, Lactobacillus, Lactococcus (optionally L. lactis), Leuconostoc, Pediococcus, Streptococcus (optionally S. sanguinus), Aerococcus, Alloiococcus, Carnobacterium, Dolosigranulum, Enterococcus, Oenococcus, Tetragenococcus, Vagococcus or Weissella,
and optionally the pharmaceutical composition or formulation is administered to or applied to or on: an oral mucosa or periodontal tissue, a tongue, a gut or a colon, a sinus mucosa, a vaginal mucosa, a stomach, skin, bladder, urethral mucosa, a ureter, an ear, bronchial mucosa, a trachea, a pharynx or a lung.

In alternative embodiments, provided are methods for treating, ameliorating, preventing or reducing the severity of or slowing the progress of a periodontitis or a gingivitis, comprising: administering in an individual in need thereof, or applying to a surface in need thereof, a pharmaceutical composition or formulation as provided herein.

In alternative embodiments, provided are methods for decreasing breath odor, or for increasing the freshness of breath, comprising: administering in an individual in need thereof, or applying to a surface in need thereof, a pharmaceutical composition or formulation as provided herein.

In alternative embodiments, provided are methods for improving the cosmetic appearance of teeth, comprising: administering in an individual in need thereof, or applying to a tooth surface in need thereof, a pharmaceutical composition or formulation as provided herein.

In alternative embodiments, provided are methods for restoring a normal oral microbiome in a smoker, a vaper or a tobacco chewer, or for increasing the amount of Streptococcus sanguinis and/or Staphylococcus epidermidis or any bacterial strain as provided herein or as used in methods as provided herein in an in vivo environment, optionally an oral environment, comprising: administering in an individual in need thereof, a pharmaceutical composition or formulation as provided herein.

In alternative embodiments of methods as provided herein: the pharmaceutical composition or formulation is contained in or on (optionally, coated on) or delivered using a delivery device, a syringe, vial, cartridge, an implant, a dressing or patch, a hydrogel, an implantation device, a mouthguard, an orthodontic appliance, a chip or slow release chip, a filament, a needle, a bandage, a gauze, or equivalent thereof.

In alternative embodiments, provided are a delivery device, a syringe, vial, cartridge, an implant, a mesh, a fiber, a plug, a tube, a coating, a rod, a dressing or patch, a tray or oral appliance, a hydrogel, a chip or slow release chip, a filament, a needle, a bandage, a gauze, or equivalent thereof, comprising or having stored or carried therein a pharmaceutical composition or formulation as provided herein.

In alternative embodiments, provided are uses of a delivery device, a syringe, vial, cartridge, an implant, a mesh, a fiber, a plug, a tube, a coating, a rod, a dressing or patch, a tray or oral appliance, a hydrogel, a chip or slow release chip, a filament, a needle, a bandage, a gauze, or equivalent thereof, as provided herein for use in:
treating, ameliorating, preventing or reducing the growth of: a biofilm, optionally a biofilm in an oral environment, optionally a biofilm growing on or adherent to a tooth, a medical device (optionally a bone implant, a pin, a mesh, a stent or an artificial valve), an implant or a prosthetic (optionally an ocular lens), optionally an oral implant or oral prosthetic; or, a microbial colony found in a biofilm;
treating, ameliorating, preventing or reducing the growth of, a biofilm-associated microorganism, or converting the biofilm-associated microorganism to a planktonic state;
treating, ameliorating, preventing or reducing the severity of or slowing the progress of a periodontitis or a gingivitis,
decreasing breath odor, or for increasing the freshness of breath, or
restoring a normal oral microbiome in a smoker, a vaper or a tobacco chewer, or for increasing the amount of Streptococcus sanguinis and/or Staphylococcus epidermidis or any bacterial strain as provided herein or as used in a method as provided herein in an in vivo environment, optionally an oral environment.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates the results of a culture plate disk zone sensitivity assay where C. matruchotii CCUG 47160 (CM47160) was challenged with paper disks soaked with biocide supernatant, as discussed in detail in Example 1, below.

FIG. 2 illustrates an EGEN-14 overnight (O/N) culture was set up using 10 uL of stock in 5 mL of BHI broth, where 50 uL of the resulting culture after overnight incubation was added to paper disks and used to confront an SH46005 (Staphylococcus hominis) lawn, as described in detail in Example 2, below.

FIG. 3 illustrates EGEN-68 O/N culture was set up using 10 uL of stock in 5 mL of BHI broth. 50 uL of the resulting culture after overnight incubation was added to paper disks and used to confront a SH46005 lawn, as described in detail in Example 2, below.

FIG. 4 illustrates EGEN-14/68 O/N culture was set up using 5 uL of each stock in 5 mL of BHI broth. 50 uL of the resulting culture after overnight incubation was added to paper disks and used to confront a SH46005 lawn, as described in detail in Example 2, below.

FIG. 5 illustrates a lawn of CD60194B was confronted with 50 uL disks of EGEN 14/68 coculture (top), EGEN-68 (bottom left), EGEN-14 (bottom right), and a bleach control, as described in detail in Example 2, below.

FIG. 6 illustrates a lawn of CD37331 was confronted with 50 uL disks of EGEN 14/68 coculture (top), EGEN-68 (bottom left), EGEN-14 (bottom right), and a bleach control, as described in detail in Example 2, below.

FIG. 7 illustrates a lawn of CD51244 was confronted with 50 uL disks of EGEN 14/68 coculture (top), EGEN-68 (bottom left), EGEN-14 (bottom right), and a bleach control, as described in detail in Example 2, below.

FIG. 8 illustrates a lawn of CM47160 was confronted with 50 uL disks of EGEN 14/68 coculture (top), EGEN-68 (bottom left), EGEN-14 (bottom right), and a bleach control, as described in detail in Example 2, below.

FIG. 9 illustrates a lawn of CM46620 was confronted with 50 uL disks of EGEN 14/68 coculture (top), EGEN-68 (bottom left), EGEN-14 (bottom right), and a bleach control, as described in detail in Example 2, below.

FIG. 10 illustrates a lawn of CM34319 was confronted with 50 uL disks of EGEN 14/68 coculture (top), EGEN-68 (bottom left), EGEN-14 (bottom right), and a bleach control, as described in detail in Example 2, below.

FIG. 11 illustrates a lawn of CM2754T was confronted with 50 uL disks of EGEN 14/68 coculture (top), EGEN-68 (bottom left), EGEN-14 (bottom right), and a bleach control, as described in detail in Example 2, below.

FIG. 12 illustrates a lawn of CM37876 was confronted with 50 uL disks of EGEN 14/68 coculture (top), EGEN-68 (bottom left), EGEN-14 (bottom right), and a bleach control, as described in detail in Example 2, below.

FIG. 13 illustrates a lawn of PM138 was confronted with 50 uL disks of EGEN 14/68 coculture (top), EGEN-68 (bottom left), EGEN-14 (bottom right), and a bleach control, as described in detail in Example 2, below.

FIG. 14 illustrates a lawn of PM33828 was confronted with 50 uL disks of EGEN 14/68 coculture (top), EGEN-68 (bottom left), EGEN-14 (bottom right), and a bleach control, as described in detail in Example 2, below.

FIG. 15 illustrates a lawn of SG27308 was confronted with 50 uL disks of EGEN 14/68 coculture (top), EGEN-68 (bottom left), EGEN-14 (bottom right), and a bleach control, as described in detail in Example 2, below.

FIG. 16 illustrates a lawn of SG35762 was confronted with 50 uL disks of EGEN 14/68 coculture (top), EGEN-68 (bottom left), EGEN-14 (bottom right), and a bleach control, as described in detail in Example 2, below.

FIG. 17 illustrates a lawn of Smu35023 was confronted with 50 uL disks of EGEN 14/68 coculture (top), EGEN-68 (bottom left), EGEN-14 (bottom right), and a bleach control, as described in detail in Example 2, below.

FIG. 18 illustrates a lawn of Smu33704 was confronted with 50 uL disks of EGEN 14/68 coculture (top), EGEN-68 (bottom left), EGEN-14 (bottom right), and a bleach control, as described in detail in Example 2, below.

Like reference symbols in the various drawings indicate like elements.

### DETAILED DESCRIPTION

In alternative embodiments, provided are compositions, including products of manufacture, pharmaceutical compositions and kits, and methods, for treating, ameliorating, preventing or reducing the growth of, a biofilm, such as a biofilm in an oral environment, such as a biofilm growing on or adherent to a tooth or an oral prosthetic, or microbial colonies found in biofilms. The biofilms can be present on any surface, including on medical or dental devices or implants or any surface, including an industrial surface. In alternative embodiments, the tooth surface can be enamel, dentin or a root canal. In alternative embodiments, the surface can be on eyeglasses, contact lenses, a door, door handle, sink, toilet, faucet, furniture and the like, and the industrial surface can be a fermentor, a heating system, a water heater, an air conditioning unit, a valve or a tube.

In alternative embodiments, provided are compositions, including products of manufacture and kits, and methods, for treating, ameliorating, preventing or reducing the severity of an infection, including infections, including nosocomial infections, associated with, exacerbated by or caused to microorganism found in or associated with biofilms.

In alternative embodiments, provided are biocide formulations and methods of using them for: treating, ameliorating or preventing a microbial growth, particularly a pathogenic microbial growth, and/or a biofilm formation. In alternative embodiments, biocide formulations as provided herein and as used in methods as provided herein comprise a bacteriocin as an active agent, where the bacteriocin can comprise a small molecule, proteinaceous or a peptidic toxin produced by a bacteria, and where the bacteriocin can act as a bacteriostatic (to inhibit the growth of bacteria) or a bactericidal agent. In alternative embodiments, bacteriocins as provided herein and as used in methods as provided herein are derived from a Staphylococcus or a lactic acid bacteria (LAB), including bacteriocins produced by Bifidobacterium, Lactobacillus, Lactococcus, Leuconostoc, Pediococcus, Streptococcus (including S. sanguinus), Aerococcus, Alloiococcus, Carnobacterium, Dolosigranulum, Enterococcus, Oenococcus, Tetragenococcus, Vagococcus and Weissella.

In alternative embodiments, compositions, including products of manufacture and kits, and methods as provided herein are used to inhibit the formation of or neutralize biofilm formation, including inhibiting or slowing the formation of or attachment of early colonizing microorganisms to a surface (for example, a tooth or any hard surface) and the subsequent development of multiple layers of cells of the same or unrelated bacterial species. In alternative embodiments, compositions, including products of manufacture and kits, and methods as provided herein are used to inhibit the formation of or neutralize dental plaque, includes any biofilm formation containing microbes including bacteria in the oral cavity, including on the surface of the teeth. In alternative embodiments, compositions, including products of manufacture and kits, and methods as provided herein are used to inhibit the growth of or the progressive development of any biomass, cell output, cell abundance and/or cell continuation of any microorganism such as bacteria, algae, diatoms, plankton, and fungi.

In alternative embodiments, compositions, including products of manufacture and kits, and methods as provided herein are used to treat, ameliorate, prevent or slow the progress of gingivitis, periodontitis or the formation of a periodontal or gingival biofilm.

In alternative embodiments, compositions, including products of manufacture and kits, and methods as provided herein are used to treat, ameliorate, prevent diseases or pathology caused by, to or slow the growth of, any biofilm-associated microorganism, including Actinomycetes, Corynebacterium, Streptococcus (including S. sanguinus), Haemophilus, Aggregribacter, Porphyromonas, Neisseria, Capnocytophaga, Fusobacterium and/or Leptotrichia.

In alternative embodiments, compositions, including products of manufacture and kits, and methods as provided herein are formulated for application to, or are applied to: an oral mucosa or periodontal tissue, a tongue, a gut or a colon, a sinus mucosa, a vaginal mucosa, a stomach, skin, bladder, urethral mucosa, a ureter, an ear, bronchial mucosa, a trachea, a pharynx or a lung, or any other microbiome space or microbiome-comprising tissue.

In alternative embodiments, provided are biocide formulations and methods of using them for decreasing breath odor, or for increasing the freshness of breath or as or in a breath freshener.

In alternative embodiments, provided are biocide formulations and methods of using them for improving the cosmetic appearance of teeth.

Streptococcus sanguinis Bacterial Strains

In alternative embodiments, provided are products of manufacture, pharmaceutical compositions or formulations comprising: a Streptococcus sanguinis bacterial strain comprising (or having contained therein) a 16S rRNA gene sequence with at least about 80%, 85%, 90%, 95%, 96%, 97%, 98%, 99% or 99.5% or more, or between about 95% and 99.8%, or between about 90% and 99.9%, or between about 85% and 100%, percent sequence identity to the sequence of SEQ ID NO:1; or, at least about 80%, 85%, 90%, 95%, 96%, 97%, 98%, 99% or 99.5% or more, or between about 95% and 99.8%, or between about 85% and 100%, or complete percent sequence identity to a 16S rRNA gene of a Streptococcus sanguinis SK36 strain (a strain having complete (100%) percent sequence identity to SEQ ID NO: 1 is also called EGEN14), wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

SEQ ID NO: 1:
GGTATAGGTGATATACTAATATAGTTGTCGCGAAAGACAAAGCCC
TTTGAAAACTGAACAAGACGAACCAAGTGCAGGGTGACATAGAGA
TATGTAACCTGTCAAAAAAACGAAAATAAATCTGTCAGTGGACAG
TAATGAGTGCGAACTCAAACTTTTTAATGAGAGTTTGATCCTGGC
TCAGGACGAACGCTGGCGGCGTGCCTAATACATGCAAGTAGAACG
CTGAAGAGAGGAGCTTGCTCTTCTTGGATGAGTTGCGAACGGGTG
AGTAACGCGTAGGTAACCTGCCTGGTAGCGGGGGATAACTATTGG
AAACGATAGCTAATACCGCATAAAATTGATTATTGCATGATAATT
AATTGAAAGATGCAACTGCATCACTACCAGATGGACCTGCGTTGT
ATTAGCTAGTTGGTGAGGTAACGGCTCACCAAGGCGACGATACAT
AGCCGACCTGAGAGGGTGATCGGCCACACTGGGACTGAGACACGG
CCCAGACTCCTACGGGAGGCAGCAGTAGGGAATCTTCGGCAATGG
GGGGAACCCTGACCGAGCAACGCCGCGTGAGTGAAGAAGGTTTTC
GGATCGTAAAGCTCTGTTGTAAGAGAAGAACGGGTGTGAGAGTGG
AAAGTTCACACTGTGACGGTATCTTACCAGAAAGGGACGGCTAAC
TACGTGCCAGCAGCCGCGGTAATACGTAGGTCCCGAGCGTTGTCC
GGATTTATTGGGCGTAAAGCGAGCGCAGGCGGTTAGATAAGTCTG
AAGTTAAAGGCTGTGGCTTAACCATAGTATGCTTTGGAAACTGTT
TAACTTGAGTGCAGAAGGGGAGAGTGGAATTCCATGTGTAGCGGT
GAAATGCGTAGATATATGGAGGAACACCGGTGGCGAAAGCGGCTC
TCTGGTCTGTAACTGACGCTGAGGCTCGAAAGCGTGGGGAGCAAA
CAGGATTAGATACCCTGGTAGTCCACGCCGTAAACGATGAGTGCT
AGGTGTTAGGCCCTTTCCGGGGCTTAGTGCCGCAGCTAACGCATT
AAGCACTCCGCCTGGGGAGTACGACCGCAAGGTTGAAACTCAAAG
GAATTGACGGGGGCCCGCACAAGCGGTGGAGCATGTGGTTTAATT
CGAAGCAACGCGAAGAACCTTACCAGGTCTTGACATCCCTCTGAC
CGCTCTAGAGATAGAGTTTTCCTTCGGGACAGAGGTGACAGGTGG
TGCATGGTTGTCGTCAGCTCGTGTCGTGAGATGTTGGGTTAAGTC
CCGCAACGAGCGCAACCCCTATTGTTAGTTGCCATCATTGAGTTG
GGCACTCTAGCGAGACTGCCGGTAATAAACCGGAGGAAGGTGGGG
ATGACGTCAAATCATCATGCCCCTTATGACCTGGGCTACACACGT
GCTACAATGGCTGGTACAACGAGTCGCAAGCCGGTGACGGCAAGC
TAATCTCTGAAAGCCAGTCTCAGTTCGGATTGTAGGCTGCAACTC
GCCTACATGAAGTCGGAATCGCTAGTAATCGCGGATCAGCACGCC
GCGGTGAATACGTTCCCGGGCCTTGTACACACCGCCCGTCACACC
ACGAGAGTTTGTAACACCCGAAGTCGGTGAGGTAACCGTAAGGAG
CCAGCCGCCTAAGGTGGGATAGATGATTGGGGTGAAGTCGTAACA
AGGTAGCCGTATCGGAAGGTGCGGCTGGATCACCTCCTTTCTAAG
GAGTCCGTAAGGACACACGGAATGCACTTGGGTCTTGTTTAGTTT
TGAGAGGGCTATGTGGGGCCTTAGCTCAGCTGGGAGAGCGCCTGC
TTTGCACGCAGGAGGTCAGCGGTTCGATCCCGCTAGGCTCCATTA
TCTAGTTTGACTAGATAAAGAACTTGTCCATTGAAAATTGAATAC
CGATATCAAATAGTAACAAGAAAATAAACCGAAAACGCTGTGAAT
ATTAATGAGTTTAAGACTGAAAGGTCAAAAATAAGGTTAAGTTAG
TAAGGGCGCACGGTGGATGCCTTGGCACTAGGAGCCGATGAAGGA
CGTGACAAACGACGAAATGCCTCGGGGAGCTGTAAGTAAGCTTCG
ATCCGGGGGTGTCCGAATGGGGGAACCCAACAGGTTGATGCCTGT
TACCCATTTCTGTTAAGGGAATGAGGAGGAAGACGCAGTGAACTG
AAACATCTAAGTAGCTGCAGGAAGAGAAAGCAAAAGCGATTGCCT
TAGTA

Staphylococcus epidermidis Bacterial Strains

In alternative embodiments, provided are products of manufacture, pharmaceutical compositions or formulations comprising: a Staphylococcus epidermidis bacterial strain comprising (or having contained therein) a 16S rRNA gene sequence with at least about 80%, 85%, 90%, 95%, 96%, 97%, 98%, 99% or 99.5% or more, or between about 95% and 99.8%, percent, or between about 90% and 99.9%, or between about 85% and 100%, or complete (100%) sequence identity to the sequence of SEQ ID NO:2; (a strain having complete (100%) percent sequence identity to SEQ ID NO:2 is also called EGEN68), wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

SEQ ID NO: 2:
AGAGTTTGATCCTGGCTCAGGATGAACGCTGGCGGCGTGCCTAAT
ACATGCAAGTCGAGCGAACAGACGAGGAGCTTGCTCCTCTGACGT
TAGCGGCGGACGGGTGAGTAACACGTGGATAACCTACCTATAAGA
CTGGGATAACTTCGGGAAACCGGAGCTAATACCGGATAATATATT
GAACCGCATGGTTCAATAGTGAAAGACGGTTTTGCTGTCACTTAT
AGATGGATCCGCGCCGCATTAGCTAGTTGGTAAGGTAACGGCTTA
CCAAGGCAACGATGCGTAGCCGACCTGAGAGGGTGATCGGCCACA
CTGGAACTGAGACACGGTCCAGACTCCTACGGGAGGCAGCAGTAG
GGAATCTTCCGCAATGGGCGAAAGCCTGACGGAGCAACGCCGCGT
GAGTGATGAAGGTCTTCGGATCGTAAAACTCTGTTATTAGGGAAG
AACAAATGTGTAAGTAACTATGCACGTCTTGACGGTACCTAATCA
GAAAGCCACGGCTAACTACGTGCCAGCAGCCGCGGTAATACGTAG
GTGGCAAGCGTTATCCGGAATTATTGGGCGTAAAGCGCGCGTAGG
CGGTTTTTTAAGTCTGATGTGAAAGCCCACGGCTCAACCGTGGAG
GGTCATTGGAAACTGGAAAACTTGAGTGCAGAAGAGGAAAGTGGA
ATTCCATGTGTAGCGGTGAAATGCGCAGAGATATGGAGGAACACC
AGTGGCGAAGGCGACTTTCTGGTCTGTAACTGACGCTGATGTGCG
AAAGCGTGGGGATCAAACAGGATTAGATACCCTGGTAGTCCACGC
CGTAAACGATGAGTGCTAAGTGTTAGGGGGTTTCCGCCCCTTAGT
GCTGCAGCTAACGCATTAAGCACTCCGCCTGGGGAGTACGACCGC
AAGGTTGAAACTCAAAGGAATTGACGGGGACCCGCACAAGCGGTG
GAGCATGTGGTTTAATTCGAAGCAACGCGAAGAACCTTACCAAAT
CTTGACATCCTCTGACCCCTCTAGAGATAGAGTTTTCCCCTTCGG
GGGACAGAGTGACAGGTGGTGCATGGTTGTCGTCAGCTCGTGTCG
TGAGATGTTGGGTTAAGTCCCGCAACGAGCGCAACCCTTAAGCTT
AGTTGCCATCATTAAGTTGGGCACTCTAAGTTGACTGCCGGTGAC
AAACCGGAGGAAGGTGGGGATGACGTCAAATCATCATGCCCCTTA
TGATTTGGGCTACACACGTGCTACAATGGACAATACAAAGGGTAG
CGAAACCGCGAGGTCAAGCAAATCCCATAAAGTTGTTCTCAGTTC
GGATTGTAGTCTGCAACTCGACTATATGAAGCTGGAATCGCTAGT
AATCGTAGATCAGCATGCTACGGTGAATACGTTCCCGGGTCTTGT
ACACACCGCCCGTCACACCACGAGAGTTTGTAACACCCGAAGCCG
GTGGAGTAACCATTTGGAGCTAGCCGTCGAAGGTGGGACAAATGA
TTGGGGTGAAGTCGTAACAAGGTAGCCGTATCGGAAGGTGCGGCT
GGATCACCT

### Bacteriocins

In alternative embodiments, bacteriocins in biocide formulations as provided herein and as used in methods as provided herein are homogenous or heterogeneous mixtures. In alternative embodiments, the bacteriocins are derived from gram-positive bacteria, including: Group I bacteriocins such as lantibiotics, where lantibiotics are peptides with a molecular weight under 2 kDa; Group II bacteriocins, which are heat-stable peptides with a molecular weight ranging from 3 to 10 kDa, wherein the majority of bacteriocins produced by Lactobacillus species belong to this group; Group III bacteriocins, which are heat-labile protein; and, Group IV bacteriocins, which have a lipid or a carbohydrate component to the protein and include for example plantaricin S and lactocin 27.

In alternative embodiments, bacteriocins in biocide formulations as provided herein and as used in methods as provided herein comprise: acidophilin, agrocin, alveicin, aureocin, bulgarican, carnocin, caseicin, colicin, curvaticin (curvacin A), diplococcin, divercin, enterocin, enterolysin, epidermin, erwiniocin, gallidermin, glycinecin, halocin, helveticin J, lactococcin (lactococcin A), lacticin (lactacin B, lactacin F), lactocin (lactocin S, lactocin 27), leucoccin, mesentericin, nisin, pediocin (pediocin PA-1), plantaricin (plantaricin S), sakacin (sakacin P), subtilin, sulfolobicin, subtilomycin, thuricin 17, trifolitoxin, vibriocin, warnerin and/or any combination thereof.

In alternative embodiments, bacteriocins in biocide formulations as provided herein and as used in methods as provided herein are isolated from a microorganism such as a bacteria or are recombinantly produced.

In alternative embodiments, bacteriocins in biocide formulations as provided herein and as used in methods as provided herein are: bactericidal and/or bacteriostatic for any microorganism, including bacteria, that colonize or contribute to the growth or maintenance of a biofilm; able to disrupt the biofilm and/or convert the biofilm microorganism from a biofilm-inhabiting state to a planktonic state (for example, to a bacteria floating in a free state apart from a formally organized biofilm), and bacteriocins in biocide formulations as provided herein and as used in methods as provided herein can be active against any biofilm-associated microorganism (or biofilm-producing), including Actinomycetes, Corynebacterium, Streptococcus, Haemophilus, Aggregribacter, Porphyromonas, Neisseria, Capnocytophaga, Fusobacterium and/or Leptotrichia.

### Bacteriocin Production

In alternative embodiments, biocide formulations as provided herein and as used in methods as provided herein comprise a bacteriocin as an active agent, where the bacteriocin can comprise a small molecule, proteinaceous or a peptidic toxin produced by a microorganism such as a bacteria, for example, a LAB. In alternative embodiments, the bacteriocin-comprising formulation is or comprises a microbial, for example, a bacterial, growth supernatant, including a growth supernatant following fermentation or any culture conditions.

In alternative embodiments, culture or fermentation conditions are optimized for bacteriocin yield, including optimization of temperature, pH and growth nutrients. Bacterial cultures used to produce the bacteriocin can be pure or mixed, and the starting culture that is used in the culture or fermentation can comprise a lactic acid bacteria, for example, a Streptococcus species (including S. sanguinus) and/or Staphylococcus epidermidis.

In alternative embodiments, bacteriocin-comprising biocide formulations as provided herein and as used in methods as provided herein are partially purified fermentation or culture supernatants, for example, comprising proteins of only a specific size as fractionated by size exclusion chromatography or selective filtering.

In alternative embodiments, bacteriocins or bacteriocin-comprising supernatants or supernatant fractions from bacterial cultures or fermentations use as or in biocide formulations as provided herein and/or as used in methods as provided herein are derived or prepared from the culturing or fermentation of Bifidobacterium, Enterobacteriaceae, Lactobacillus, Lactococcus, Leuconostoc, Pediococcus, Streptococcus (including S. sanguinus), Aerococcus, Alloiococcus, Carnobacterium, Dolosigranulum, Enterococcus, Oenococcus, Tetragenococcus, Vagococcus and Weissella, or any species that belong to the order Lactobacilloles.

### Bacteriocin-Comprising Probiotics

In alternative embodiments, biocide formulations as provided herein and as used in methods as provided herein comprise living bacteriocin-producing or bacteriocin-secreting live, non-pathogenic bacteria, such as LAB bacteria, including Bifidobacterium, Enterobacteriaceae, Lactobacillus, Lactococcus, Leuconostoc, Pediococcus, Streptococcus (including S. sanguinus), Aerococcus, Alloiococcus, Carnobacterium, Dolosigranulum, Enterococcus, Oenococcus, Tetragenococcus, Vagococcus and Weissella, or any species that belong to the order Lactobacillus, or any combination thereof.

In alternative embodiments, the living bacteriocin-producing or bacteriocin-secreting bacteria are delivered at concentrations of from between about 1×10² to about 1×10¹², or 1×10³ to about 1×10¹¹, or 1×10⁴ to about 1×10¹⁰, or 1×10⁵ to about 1×10⁹, CFU/gram (g) of the bacterial strain with respect to a total weight of the pharmaceutical composition, or the living bacteriocin-producing or bacteriocin-secreting bacteria are present in the formulation or pharmaceutical composition, in an amount that comprises from about 1×10² to about 1×10¹², or 1×10³ to about 1×10¹¹, or 1×10⁴ to about 1×10¹⁰, or 1×10⁵ to about 1×10⁹, CFU per unit dose.

### Bacteriocin-Comprising Supernatants

In alternative embodiments, biocide formulations as provided herein and as used in methods as provided herein comprise a supernatant of a culture or fermentation of a Streptococcus sanguinis and/or Staphylococcus epidermidis bacterial strain or of a bacterial strain as provided herein or as used in a method as provided herein, wherein the supernatant is capable of secreting a bacteriocin (or any bacteriocidal compound, which optionally can be a protein, peptide or carbohydrate) capable of inhibiting the growth of a biofilm-forming bacterium or inhibiting the formation of a biofilm.

In alternative embodiments, supernatants are prepared by a batch or a continuous fermentation process, wherein a tank fermenter or batch fermenter is filled with the prepared mash of raw materials to be fermented. The temperature and pH for microbial fermentation is properly adjusted, and occasionally nutritive supplements are added to the prepared mash. The mash can be steam sterilized in a pure or mixed culture process. An inoculum of a culture of bacteria of interest, for example, a Streptococcus sanguinis and/or Staphylococcus epidermidis bacterial strain as provided herein, or bacteria as used in a method as provided herein, is added to the fermenter or batch from a separate culture vessel. A cell-free supernatant can be collected following high speed centrifugation of cells that are actively growing. Alternatively, the supernatant can be filtered to remove all or substantially all bacteria.

### Supernatant Fractionation

In alternative embodiments, biocide formulations as provided herein and as used in methods as provided herein comprise a fraction or isolate of a supernatant of a culture or fermentation of a Streptococcus sanguinis and/or Staphylococcus epidermidis bacterial strain, or a bacterial strain as provided herein or a bacterial strain used in a method as provided herein, wherein the fraction or isolate is capable of secreting a bacteriocin capable of inhibiting the growth of a biofilm-forming bacterium or inhibiting the formation of a biofilm.

In alternative embodiments, the fraction or isolate is prepared by filtration using membrane filters specific for the size of the bacteriocin. Membrane filters in the range of 3 KDa to 30 KDa can be used to obtain the desired fraction.

In alternative embodiments, a supernatant or bacterial cell culture (for example, of a Streptococcus sanguinis bacterial strain and/or Staphylococcus epidermidis bacterial strain, or of a bacterial strain as provided herein or as used in a method as provided herein), including a cell free supernatant or bacterial cell culture (for example, where substantially all bacteria have been removed from the supernatant or bacterial cell culture liquid or media) can be further treated or fractionated, for example, by column chromatography, for example, a high pressure liquid chromatograph (HPLC), where an eluate shown to be capable of inhibiting or slowing the growth of a biofilm-forming bacterium or inhibiting or slowing the formation of a biofilm is harvested and used in or for preparing a pharmaceutical composition or formulation as provided herein.

### Formulations

In alternative embodiments, pharmaceutical compositions and bacteriocin-comprising biocides as provided herein and as used in methods as provided herein are formulated as or included or contained in: liquids (for example, a saline, liquid buffer, or sterile water), gels, capsules, pills, tablets, geltabs, lozenges, powders, gums, sachets, cachets, elixirs, hydrogels or viscosity enhancing agents, suspensions, emulsions, liposomes or lipid carriers, solutions, toothpastes, mouthwashes, syrups, foods, pastes, candies, confectionaries, aerosols, lysosomes, microparticles, nanoparticles, microspheres and/or lyophilates.

In alternative embodiments, the amount of biocide in a unit dosage of a formulation as provided herein is between about 0.1 mg to 5 mg, 0.2 mg to 2.5 mg, or about 0.25, 0.50, 1.0, 1.5 or 2 mg or more.

In alternative embodiments, the concentration of biocide in single dosage of a formulation as provided herein is between about 0.1 mg to 5 mg, 0.2 mg to 2.5 mg, or about 0.25, 0.50, 1.0, 1.5 or 2 mg or more per ml or per gram (g).

In alternative embodiments, nanoparticles or microspheres used to deliver bacteriocin-comprising biocides comprise: metal nanoparticle systems, organic nanoparticles, bacteriocin scaffolding with nanofiber technology, nanospheres and the like.

In alternative embodiments, microparticles, nanoparticles or microspheres comprising the pharmaceutical compositions and bacteriocin-comprising biocides as provided herein, or as used in methods as provided herein are, are used to coat a surface, for example to coat a medical device such as an implant, for example, prosthetic, a bone implant, or a valve or stent. In alternative embodiments, pharmaceutical compositions and bacteriocin-comprising biocides as provided herein are formulated with (for example, carried or delivered within) microparticles or microspheres as described for example, in U.S. Pat. Nos. 10,538,658; 10,512,650; 10,493,173; 10,471,013; 10,376,469; 10,369,176; 10,300,019.

In alternative embodiments, pharmaceutical compositions and bacteriocin-comprising biocides as provided herein are formulated with (for example, carried or delivered within) liposomes or lipid carriers as described for example, in U.S. Pat. No. 10,532,975.

In alternative embodiments, gel formulations as provided herein comprise: modified celluloses (for example, hydroxypropyl cellulose and hydroxyethyl cellulose), carbopol homopolymers and copolymers, solvents such as diglycol monoethyl ether, alkylene glycols (for example, propylene glycol), dimethyl isosorbide, alcohols (for example, isopropyl alcohol and ethanol), isopropyl myristate, ethyl acetate, C12-C15 alkyl benzoates, mineral oil, squalane, cyclomethicone, capric/caprylic triglycerides, or combinations thereof.

In alternative embodiments, emulsifiers as provided herein comprise: acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl mono stearate, sodium citrate dehydrate, sodium lauryl sulfate, sorbitan esters, stearic acid, sunflower oil, tragacanth, triethanolamine and xanthan gum, or any combination thereof.

In alternative embodiments, hydrogels or viscosity enhancing agents as provided herein comprise: methylcellulose, hydroxyethylcellulose, hydroxypropyl-methylcellulose, or any combination thereof.

### Formulation Additives

In alternative embodiments, compositions, including products of manufacture, pharmaceutical compositions, biocide formulations and kits as provided herein and as used in methods as provided herein are, further comprise or comprise use of additional agents such as additional biofilm disrupting agents, stabilizers, enzymes, surfactants (including biosurfactants), a natural product, antibiotics or antimicrobials, complexing agents, buffers, peroxide, flavoring agents, preservatives, a tissue (for example, gingival or periodontal tissue) penetration enhancer, a pharmaceutically acceptable rate modifying agent, a sustained-release polymer, anti-inflammatory agents, immune-suppressive agents, immune-stimulatory agents, dentinal desensitizers, an odor masking agent, and the like.

In alternative embodiments, enzymes used in compositions, including products of manufacture and pharmaceutical compositions as provided herein, or used in or with methods as provided herein, comprise biofilm disrupting enzymes such as, for example, a proteinase, an amylase, a lyase, a deoxyribonuclease (DNase), optionally dornase alpha, or PULMOZYME™, an alginase, a lyase or a glycoside hydrolase (optionally dispersin B), or any combination thereof.

In alternative embodiments, stabilizers used in compositions, including products of manufacture and pharmaceutical compositions as provided herein, or used in or with methods as provided herein, comprise compositions able to protect proteins and increase the shelf life of the product, such as highly water-soluble sodium and/or potassium salts.

In alternative embodiments, antibiotics or antimicrobials used in compositions, including products of manufacture and pharmaceutical compositions as provided herein, or used in or with methods as provided herein, comprise algicides, fungicides and/or non-oxidizing biocides. In alternative embodiments, the antibiotic or antimicrobials comprise at least one of: alkyldimethylammonium chloride (as used for example, in U.S. Pat. Nos. 5,827,503; 5,753,711), a 4-dedimethylaminotetracycline compound or derivative as described for example, in U.S. Pat. No. 6,506,740, a nitroimidazole, a paromomycin, an iodoquinol, a doxycycline, norfloxacin, ciprofloxacin, levofloxacin, vancomycin, rifaximin, streptomycin or neomycin secnidazole, nitazoxanide, furazolidone, azithromycin, clarithromycin, gentamicin, vancomycin, rifaximin, rifabutin, rifampicin, nitroimidazole, streptomycin, erythromycin, roxithromycin, DEA-CP, bismuth thiol, bismuth subcitrate; bismuth subsalicylate; bismuth ethanondiothol, bismuth dimercaprol, bismuth dimercapropranol and mixtures and combinations thereof.

In alternative embodiments, compositions, including products of manufacture and pharmaceutical compositions as provided herein, or used in or with methods as provided herein, comprise an iodine, povidone, povidone-iodine (PI) (or WOKADINE™, PYODINE™, BETADINE™).

In alternative embodiments, compositions, including products of manufacture and pharmaceutical compositions as provided herein, or used in or with methods as provided herein, comprise an anti-persister cell therapy to activate resister bacteria in biofilm matrix, which can comprise pyruvate, a sugar and/or a polyol (optionally a sugar alcohol), and optionally the sugar and/or a polyol comprises mannitol, glucose or fructose or any combination thereof.

In alternative embodiments, compositions, including products of manufacture and pharmaceutical compositions as provided herein, or used in or with methods as provided herein, comprise vitamins, for example, Vitamin C or L-ascorbic acid.

In alternative embodiments, compositions, including products of manufacture and pharmaceutical compositions as provided herein, or used in or with methods as provided herein, comprise or use other biofilm degrading or anti-biofilm substances such as: a 2-amino-imidazole such as oroidin, 2-amino-imidazole/triazole (2-AIT), or ageliferin, a fatty acid such as cis-2-decenoic acid (C2DA), S-Nitrosoglutathione (GSNO), S-Nitroso-N-acetylpenicillamine (SNAP), Gc protein-derived macrophage activating factor (GcMAF), Acyldepsipeptide or cyclic acyldepsipeptide (ADEP), DEA NONOate-Cephalosporin Prodrug (DEACP), N-acetylcysteine, dispersin, ribonucleic-acid-III inhibiting peptide (RIP), Salvadora persica extracts, competence-stimulating peptide (CSP) patulin (PAT), penicillic acid (PA)/EDTA, cathelicidin-derived peptides, small lytic peptide PTP-7 (see for example, Kharidia (2011) J. Microbiol. 49(4):663-8), nitric oxide, cys-2-decenoic acid, sodium nitroprusside, s-nitroso-1-glutathione (GSNfaO), s-nitroso-N-acetylpenicillamine (SNAP), chlorhexidine, a nanoemulsion, a lytic bacteriophage, a lactoferrin, a xylitol hydrogel, a synthetic iron chelator, a cranberry component, a curcumin, an acetyl-11-keto-boswellic acid (AKBA), a barley coffee (BC) component, a silver nanoparticle, a metallic silver or an ionic silver, a probiotic (for example, Bacillus), sinefungin, N-acetyl-cysteine, S-adenosylmethionine, S-adenosyl-homocysteine, a Delisea furanone, a N-sulfonyl homoserine lactone, iron or ionic silver salts (which can inhibit biofilm formation, and permit antibiotics to be more active), arsenicals, selenium, titanium dioxide, gallium nitrate, chlorine dioxide, nitrofurantoin ((E)-1-[(5-nitro-2-furyl) methylidene-amino] imidazolidine-2,4-dione), zaragozic acid, norspermidine, AA-861, trehalase, parthenolide, rhamnolipid, lipoic acid, kojic acid, picolinic acid, an alcohol such as ethanol, hydrogen peroxide, hydrochloric acid, formaldehyde or luminal formalin in low concentrations, ozonated water, hydrogenated water, super-oxidized aqueous solution, nitrofurantoin (for example, MACROBID™), hexamine hippurate (for example, HIPREX™), potassium hydroxide, mercuric chloride, iodine or povidone-iodine (PI) (or WOKADINE™, PYODINE™, BETADINE™), and/or disodium EDTA, or any combination thereof.

In alternative embodiments, compositions, including products of manufacture and pharmaceutical compositions as provided herein, or used in or with methods as provided herein, comprise or use bacteriophage (phage), for example, a phage directed against a bacterium found in a biofilm.

In alternative embodiments, compositions, including products of manufacture and pharmaceutical compositions as provided herein, or used in or with methods as provided herein, comprise or use natural products or compounds, for example, plant derived compounds, for example, ellagic acid or ellagic acid derivatives, tea-tree oils, cinnamaldehyde, chelerythrine, sanguinarine, dihydroxybenzofuran and/or proanthocyanidin, or any combination thereof.

In alternative embodiments, the preservative comprises: sugar alcohols (for example, sorbitol and mannitol), ethanol, benzyl alcohol, isopropanol, cresol, chlorocresol, phenol, or any combination thereof.

In alternative embodiments, the tissue (for example, gingival or periodontal tissue) penetration enhancer comprises N-methyl-2-pyrrolidone, 2-pyrrolidone, propylene glycol, dimethylformamide, dimethyl sulfoxide, caprolactam, oleic acid, decylmethylsulfoxide, 1-dodecylazacycloheptan-2-one, isopropyl myristate, hexamethylene palmitamide, hexamethylene lauramide, and other aliphatic acids, and esters, as described for example, in U.S. Pat. No. 4,975,271. The pharmaceutical compositions and biocide formulations as provided herein, or as used in methods as provided herein, can be mixed with one or more penetration enhancers and syringed or injected directly into a periodontal pocket, or administered to any desired tissue surface.

In alternative embodiments, a pharmaceutical compositions and biocide formulations as provided herein, or as used in methods as provided herein, are formulated with a pharmaceutically acceptable rate modifying agent, for example, as described in EP 0537559 A1, describing a liquid composition suitable for formation of a controlled release implant for use in a patient comprising: a pharmaceutically acceptable, biodegradable thermoplastic polymer that is substantially insoluble in water or body fluids; an organic solvent that is miscible or dispersible in water or body fluids; a pharmaceutically acceptable rate modifying agent; and a biologically active material, which can be a pharmaceutical compositions and biocide formulations as provided herein. The rate modifying agent can be more hydrophobic than the organic solvent, and the rate modifying agent can comprise: an ester of a mono-, di-, or tricarboxylic acid, a polyhydroxy alcohol, a fatty acid, an ester of glycerol, a sterol, or a higher alkyl alcohol.

In alternative embodiments, a pharmaceutical compositions and biocide formulations as provided herein, or as used in methods as provided herein, are formulated with a sustained-release polymer, which can comprise for example, polyacrylate, polymethacrylate, cellulose derivatives, ethylcellulose, hydroxypropylmethyl cellulose, cellulose acetate phthalate, polysaccharide, guar gum, pectin, alginic acid and salts thereof, xanthan gum, gum tragacanth, gum arabic, starch, chitin, chitosan, proteins, polyamino acids, polypeptides, gelatin, polyglycolic acid, polylactic acid, polyglycolic-polylactic copolymers, cross-linked polysaccharides, and/or cross-linked proteins, as described for example, in U.S. Pat. No. 6,197,331.

In alternative embodiments, a pharmaceutical compositions and biocide formulations as provided herein, or as used in methods as provided herein, are formulated as described in U.S. Pat. Nos. 4,764,377 and 4,892,736, describing formulating with a polymeric matrix such as ethylene vinyl acetate copolymer, which can be place onto a desired surface, for example, directly into a periodontal pocket.

### Delivery Mechanisms and Products of Manufacture

In alternative embodiments, products of manufacture, compositions, pharmaceutical compositions and biocide formulations as provided herein and as used in methods as provided herein are delivered to the biofilm or any desired surface (for example, an intra-oral surface), for example, delivered to a tooth (for example, enamel, root surface or cementum), gum (or any soft tissue) and/or bone surface, for example, delivered to a periodontal pocket, using a delivery device or product of manufacture such as, for example, a syringe, tray or oral appliance (for example, as described in USPNs10,507,093; 8,585,406; 8,113,837), a vial, a cartridge, an implant, a gauze, a dressing or patch (for example, an oral patch as described for example, in U.S. Pat. No. 6,197,331), a hydrogel, a chip or slow release chip, a filament, a needle, an implant (for example, an intra-oral or bone implant) or implantation device (for example, as described in U.S. Pat. No. 9,662,274, or U.S. Pat. Nos. 5,702,716, 5,945,115, describing a biodegradable and/or bioerodible microporous, solid polymer matrix; or USPN 5,324,51, describing a biodegradable and/or bioerodible microporous, solid or gelatinous polymer matrix; or U.S. Pat. No. 4,938,763, describing a biodegradable polymer for use in providing syringeable, in-situ forming, solid biodegradable implants).

In alternative embodiments, products of manufacture, compositions, pharmaceutical compositions and biocide formulations as provided herein and as used in methods as provided herein are formulated with or in, or are delivered to a desired site or tissue in vivo, for example, a periodontal tissue or pocket, using microparticles or microcapsules and described for example, in U.S. Pat. Nos. 5,500,228; 5,236,355; and, 5,366,733, describing use of microcapsules that can be directly inserted or injected to a periodontal pocket, where the microcapsules can be made of or comprise polyglycolide, poly (1-lactide), poly (dl-lactide), polyglycolide-co-dl-lactide), poly(glycolide-1-lactide), poly (alphahydroxybutyric acid), poly (orthoesters), poly (p-dioxanone), block copolymers of polyglycolide, trimethylene carbonate and/or polyethylene oxide, where the microparticles can be embedded in a biocompatible and biodegradable polymer comprising a silicone, which can be octamethylcyclotetrasiloxane, decamethylcyclopentasiloxane, or hexamethyldisiloxane), a mouthguard, an orthodontic appliance, or equivalents.

In alternative embodiments, products of manufacture, compositions, pharmaceutical compositions and biocide formulations as provided herein and as used in methods as provided herein are delivered to the biofilm or desired surface using a tray (for example, a periodontal interdental tray as described in U.S. Pat. No. 8,439,676) or a preformed carrier device. In alternative embodiments, compositions, pharmaceutical compositions and biocide formulations as provided herein and as used in methods as provided herein are delivered to the biofilm or desired surface as a spray or aerosol.

In alternative embodiments, the products of manufacture, device is a syringe or oral drug delivery device as described, for example, in U.S. Pat. No. 5,013,553, describing a drug delivery device that can be inserted subgingivally, for example, in a periodontal pocket, to locally deliver the drug, and where the drug can be slowly released when the drug is formulated in a bioerodable and biocompatible material.

In alternative embodiments, products of manufacture, compositions, pharmaceutical compositions and biocide formulations as provided herein and as used in methods as provided herein are delivered to the biofilm or desired surface using devices and/or compositions as described in U.S. Pat. No. 5,077,049, which describes placing a liquid solution of a biodegradable, water-coagulable, thermoplastic polymer and a water miscible non-toxic organic solvent (with a pharmaceutical composition or a biocide formulation as provided herein) into a periodontal pocket; wherein said organic solvent dissipates into periodontal fluids, and the biodegradable, water-coagulable, thermoplastic polymer coagulates to form an in situ solid biodegradable implant, and the dissipation of solvent can create pores within said solid biodegradable implant having sizes in the range of about 3 to 500 microns; and the solid biodegradable implant can have a porosity in the range of 5 to 95% provided by the pore.

In alternative embodiments, the products of manufacture or device is a syringe or oral drug delivery device as described, for example, in U.S. Pat. Nos. 6,682,348; 7,699,609, describing apparatus for delivering formulations directly into a periodontal pocket.

In alternative embodiments, products of manufacture or implants as provided herein are or comprise injectable biodegradable polymeric matrix implants, for example, based on poly(lactic-co-glycolic acid), or lactic acid, glycolic acid and/or their copolymers for sustained release as described in for example, U.S. Pat. No. 5,620,700; or as described in U.S. Pat. Nos. 6,143,314; 6,673,767; 6,331,311; 5,770,231, 7,118,763, or 10,300,019 (describing biodegradable microparticles for sustained release).

In alternative embodiments, hydrogels comprise biodegradable single-phase cohesive hydrogel compositions as described for example, in U.S. Pat. No. 9,919,076, or an injectable tissue adhesive hydrogel as described for example, in U.S. patent application publication 20170281781A1.

### Products of Manufacture and Kits

Provided are products of manufacture and kits for practicing methods as provided herein; and optionally, products of manufacture and kits can further comprise formulations and/or bacterial strains as provided herein, and/or instructions for practicing methods as provided herein.

### Treatment Regimens

Provided are pharmaceutical compositions and formulations, products of manufacture and kits for treating, ameliorating, preventing or reducing the severity of or slowing the progress of a periodontitis or a gingivitis, comprising: administering in or to an individual in need thereof, or applying to a surface in need thereof, a pharmaceutical composition or formulation as provided herein. In alternative embodiments, these methods comprise treating a periodontal pocket. In alternative embodiments, compositions and formulations are applied once a week, every other day, once a day, twice a day (bid), three times a day (tid) or more often or less often. In alternative embodiments, compositions and formulations are applied over a period of a week, a month, 2 to 11 months, a year, or more or indefinitely to treat, ameliorate, prevent or reduce the severity of or slow the progress of a periodontitis or a gingivitis,

Provided are pharmaceutical compositions and formulations, products of manufacture and kits restoring a normal oral microbiome in a smoker, a vaper or a tobacco chewer, or for increasing the amount of Streptococcus sanguinis and/or Staphylococcus epidermidis in an oral environment, comprising: administering in or to an individual in need thereof, or applying to a surface in need thereof, a pharmaceutical composition or formulation as provided herein. Treatment regimen can be the same as for treating a periodontitis or a gingivitis as provided herein.

Studies have shown that smokers have a rate of periodontitis approximately 5 times higher than nonsmokers. Scientists performed a microbiome study and observed that smokers had lower levels of Streptococcus sanguinus bacteria in the oral cavity than nonsmokers. A second study showed that the biofilm that causes periodontitis has Corynebacterium as an early colonizing species; (non-Corynebacterium) bacteria normally comprising the periodontitis biofilm first need a Corynebacterium to be present for the biofilm to be formed. Pharmaceutical compositions and formulations as provided herein are effective in reducing the growth or vitality of or killing Corynebacterium.

Our work has shown that the bacteriocins, including compositions and formulations as provided herein, produced by Streptococcus sanguinus and/or Staphylococcus epidermidis can kill or inhibit the growth of Corynebacterium. If smokers have less S. sanguinus bacteria they have less ability to kill/inhibit Corynebacterium and therefore have more foundational or early colonizing bacteria required for the periodontitis biofilm to form.

Provided are pharmaceutical compositions and formulations, products of manufacture and kits for decreasing breath odor, or for increasing the freshness of breath, or for improving the cosmetic appearance of teeth, comprising: administering in or to an individual in need thereof, or applying to a surface in need thereof, a pharmaceutical composition or formulation as provided herein. Treatment regimen can be the same as for treating a periodontitis or a gingivitis as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12%11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially”, “substantially all”, “substantially most of”, “substantially all of” or “majority of” encompass at least about 85%, 90%, 91%, 92%, 93%, 94%, 95%, 97%, 98%, 99% or 99.5%, or more of a referenced amount of a composition. For example, a formulation having substantially live and viable bacteria is a formulation where at least about 85%, 90%, 91%, 92%, 93%, 94%, 95%, 97%, 98%, 99% or 99.5%, or more of the bacteria are viable.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Exemplary Biocide and Anti-Biofilm Formulations

This example demonstrates the efficacy of exemplary biocide and anti-biofilm formulations as provided herein.

In order to show the efficacy of exemplary biocide formulations and methods as provided herein for eliminating microorganisms and preventing oral biofilm build-up, exemplary biocide formulations, including culture supernatants, were used in confrontation assay experiments.

We analyzed the bacteria present in public human microbiome datasets to confirm that our target species were, in fact, correct. Human plaque and stool metagenomics data were obtained from the Human Microbiome Project (HMP) (PMID:22699610; PMID:22699609) (BioProject accession: PRJNA48479) via Sequence Read Archive (SRA). FASTQ files were processed for quality control to trim, filter low quality, and short reads using fastp (PMID:30423086). FASTQ files were also deduplicated to remove PCR duplicates. Filtered sequences were searched (blastx) against RefSeq proteins (PMID:17130148) for a functional and taxonomic assignment using DIAMOND (PMID:25402007). The taxonomic abundance of the identified species was quantified and normalized, and the statistical analyses were performed in R (http://www.R-project.org/). Principal Component Analysis (PCA) of the taxonomic abundances in these samples showed a clear separation between stool and plaque samples by the first principal component. There was also a further separation between subgingival plaque and supragingival plaque. Analyzing the taxonomic abundances between the plaque and stool samples identified Corynebacterium matruchotii to be significantly (p-value=0.00157<<0.05) enriched in the plaque samples.

Eight strains of Corynebacterium were received from CCUG (Göteborg, Sweden) (https://www.ccug.se). They consisted of 5 Corynebacterium matruchotii strains and 3 Corynebacterium durum strains. The C. matruchotii strains consisted of CCUG 47160 isolated from human supragingival plaque in healthy periodontium, 46620T isolated from human oral calculus, and strains 37876, 34319, and 27545T isolated from oral calculus. The remaining three strains were C. durum strains and included CCUG 60194B from human parotid, 51244 from an osteomyelitis lesion, and 37331T from human sputum. All strains were cultured upon receipt, but only 4 of the C. matruchotii strains showed growth. As a result, only 7 strains that grew were used for subsequent experiments. For simplicity, each strain was referred to by the first letter of their genus and species, followed by the CCUG number that corresponds to the strain.

Plate confrontation assays were conducted using each of the strains as the lawn for its respective experiments. Of the 7 strains, only 3 strains showed zones of clearing when challenged with samples derived from healthy human plaque (challenging agent). Further experimentation with liquid confrontations revealed that the challenging agent produced clearing and lysis of cells only when confronting C. matruchotii strains, suggesting narrow-spectrum activity.

Fresh plaque samples were collected and resuspended into an eSwab kit. This resuspended material was used for confrontation assays against C. matruchotii.

Confrontation assays were performed with 31 novel isolates that were derived from the zones of clearing on agar plates made using C. matruchotii CCUG 47160 (CM47160) and challenged with the healthy human oral plaque samples. The isolates picked from streak plates of biomass collected from the zones of clearing were used as challenging agents against a lawn of CM47160. As per previous experiments, 2 mL of CM47160 was pipetted onto blood agar plates and swirled for 15 seconds. Excess supernatant was removed from the plates. Lawns were allowed to dry for at least 10 minutes. Then, 3 paper disks (samples in triplicate), each containing 50 uL of growth media from each of the 31 isolates were placed onto the lawn and incubated at 37° C. Negative and positive controls were included. Following sequential passaging and culturing the purified isolate that was responsible for the largest zones of clearing is termed Target Element 1 (TE1). Through a series of experiments, TE1 was discovered to grow best in blood agar media, rather than brain heart infusion (BHI) media. A series of dilutions 1×, 10×, 100× and 1000× were generated and used in agar based confrontation assays to gauge the lower limit of bactericidal activity of TE1.

In order to induce microbial production of TE1 antimicrobial compound (TE1 is the same as EGEN14, produced from Streptococcus sanguinis), confrontation supernatants were collected and proteins in the supernatant were extracted using the Total Protein Extraction kit (EMD Millipore catalog #2140). Protein extracts were analyzed using the Agilent High Sensitivity Protein 250™ kit according to the manufacturer's specifications. Protein profiles were analyzed and a 10 kDa protein was determined to be present in all extracts.

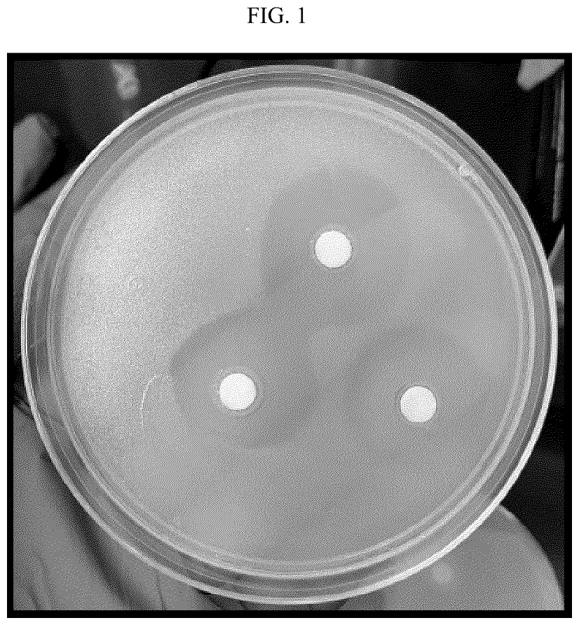

The supernatant containing the antimicrobial proteins (an exemplary biocide formulation) was used as challenging agents against a lawn of CM47160. As per previous experiments, 2 mL of CM47160 was pipetted onto blood agar plates and swirled for 15 seconds. Excess supernatant was extracted from the plate and placed back into the culture tube. Lawns were allowed to dry for at least 30 minutes. Then, 50 uL of the supernatant the undiluted isolate was added onto three paper disks. The disks were flipped and placed onto the lawn using sterilized tweezers and incubated at 37 degrees Celsius. Negative and Positive controls were performed in the same manner using blank paper disks or disks containing 50 uL of 10% bleach, respectively. The lawns were observed at 24-hour intervals. After the first 24 hour observation period, zones of clearing were evident around the paper disk inoculated with the biocide supernatant with no change to the control groups. By the 48 hour interval, the plates all showed significant zones of clearing, as illustrated in FIG. 1, with no noticeable change in the controls.

### Example 2: Exemplary Biocide and Anti-Biofilm Formulations

This example demonstrates the efficacy of exemplary biocide and anti-biofilm formulations as provided herein.

In order to show the efficacy of exemplary biocide formulations and methods as provided herein for eliminating microorganisms and preventing oral biofilm build-up, exemplary biocide formulations, including culture supernatants, were used in confrontation assay experiments, where zones of clearance signify antibacterial activity of compounds moving from the disk to the culture lawn.

Using protocols as generally described in Example 1, challenge assays were conducted where: EGEN68 was used to challenge a lawn of Staphylococcus hominis (called SH46005); EGEN14 was used to challenge a lawn of S. hominis; and, both EGEN68 and EGEN14 are combined to challenge a lawn of S. hominis. It is clear from the photograph on slide/page 4 that when both EGEN14 and EGEN68 are used in combination a significantly greater effect is observed.

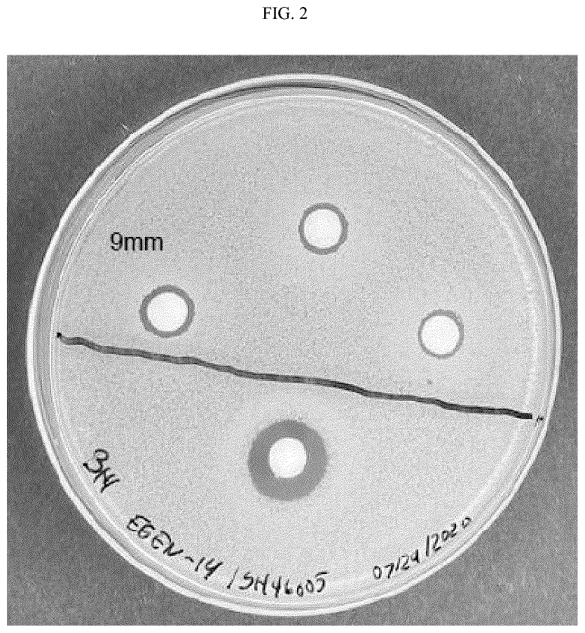

As illustrated in FIG. 2, an EGEN-14 overnight (O/N) culture was set up using 10 uL of stock in 5 mL of BHI broth. 50 uL of the resulting culture after overnight incubation was added to paper disks and used to confront an SH46005 (Staphylococcus hominis) lawn. After overnight incubation, the confrontation showed zones of clearing, with 9 mm being the largest. A bleach control was included at the bottom of the plate.

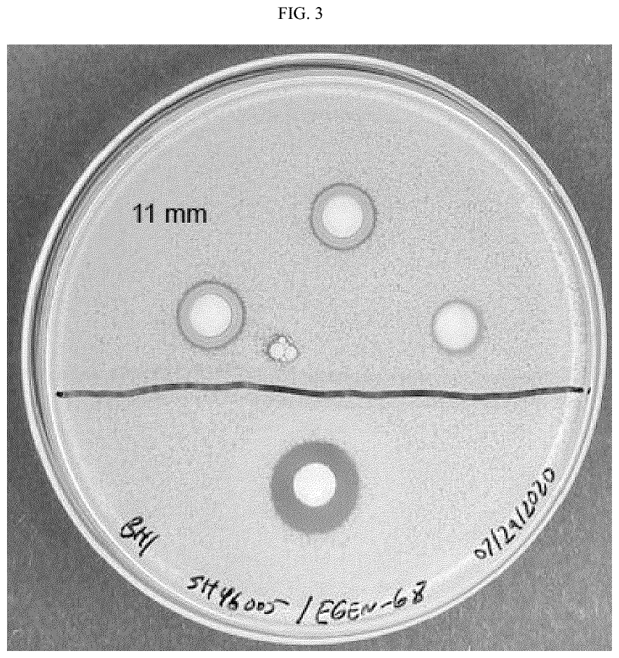

As illustrated in FIG. 3, EGEN-68 O/N culture was set up using 10 uL of stock in 5 mL of BHI broth. 50 uL of the resulting culture after overnight incubation was added to paper disks and used to confront a SH46005 (Staphylococcus hominis) lawn. After overnight incubation, the confrontation showed zones of clearing, with 11 mm being the largest. A bleach control was included at the bottom of the plate.

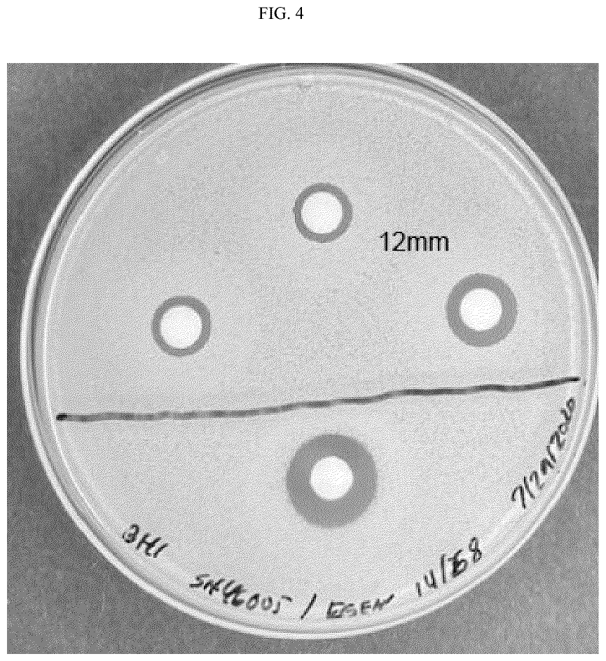

As illustrated in FIG. 4, EGEN-14/68 O/N culture was set up using 5 uL of each stock in 5 mL of BHI broth. 50 uL of the resulting culture after overnight incubation was added to paper disks and used to confront a SH46005 (Staphylococcus hominis) lawn. After overnight incubation, the confrontation showed zones of clearing, with 12 mm being the largest. A bleach control was included at the bottom of the plate.

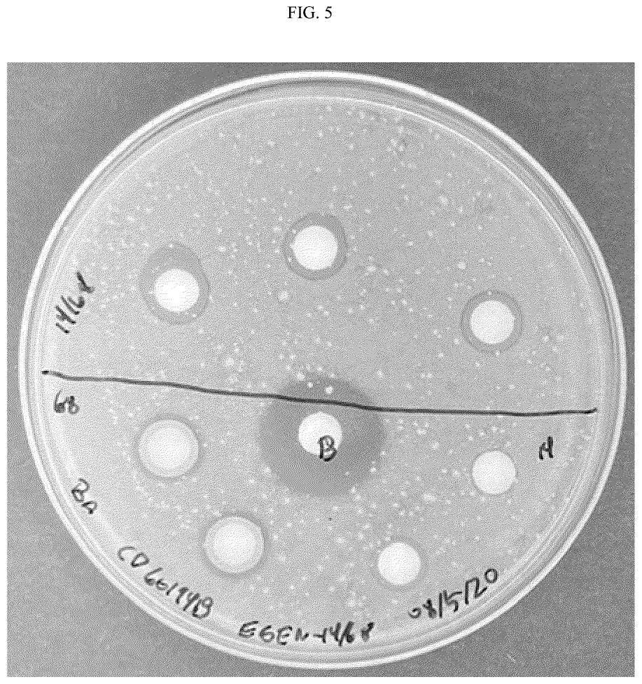

As illustrated in FIG. 5, a lawn of the Corynebacterium durum strain CD60194B was confronted with 50 uL disks of EGEN 14/68 coculture (top), EGEN-68 (bottom left), EGEN-14 (bottom right), and a bleach control. The zones of clearing were measured.

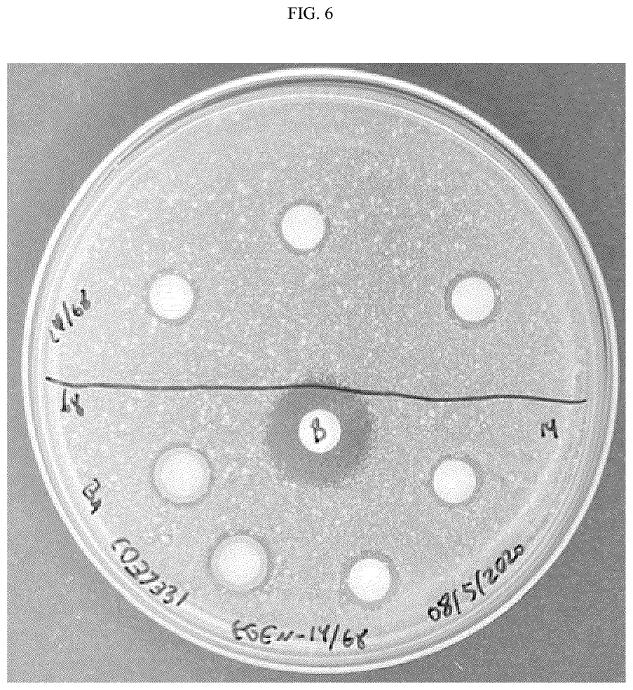

As illustrated in FIG. 6, a lawn of the Corynebacterium durum strain CD37331 was confronted with 50 uL disks of EGEN 14/68 coculture (top), EGEN-68 (bottom left), EGEN-14 (bottom right), and a bleach control.

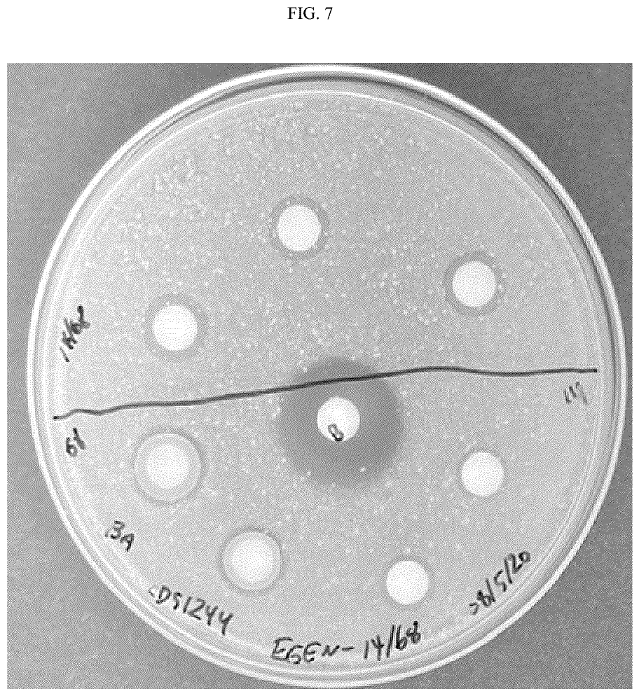

As illustrated in FIG. 7, a lawn of the Corynebacterium durum strain CD51244 was confronted with 50 uL disks of EGEN 14/68 coculture (top), EGEN-68 (bottom left), EGEN-14 (bottom right), and a bleach control.

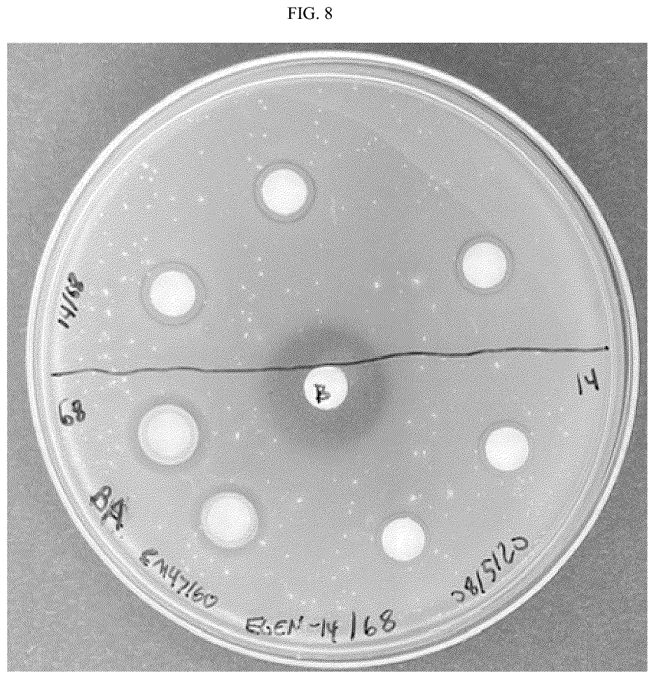

As illustrated in FIG. 8, a lawn of the Corynebacterium matruchotii strain CM47160 was confronted with 50 uL disks of EGEN 14/68 coculture (top), EGEN-68 (bottom left), EGEN-14 (bottom right), and a bleach control.

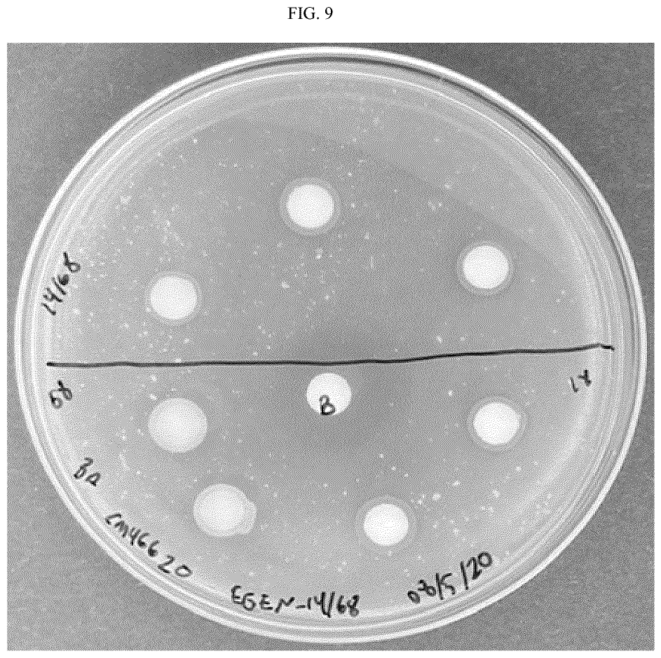

As illustrated in FIG. 9, a lawn of the Corynebacterium matruchotii strain CM46620 was confronted with 50 uL disks of EGEN 14/68 coculture (top), EGEN-68 (bottom left), EGEN-14 (bottom right), and a bleach control.

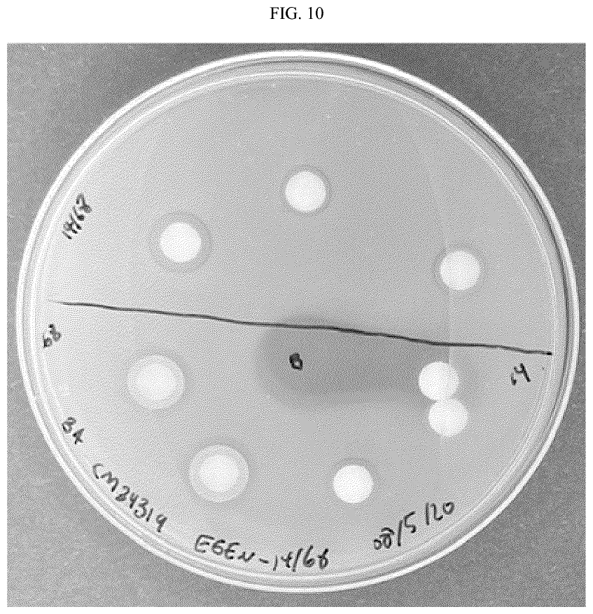

As illustrated in FIG. 10, a lawn of the Corynebacterium matruchotii strain CM34319 was confronted with 50 uL disks of EGEN 14/68 coculture (top), EGEN-68 (bottom left), EGEN-14 (bottom right), and a bleach control.

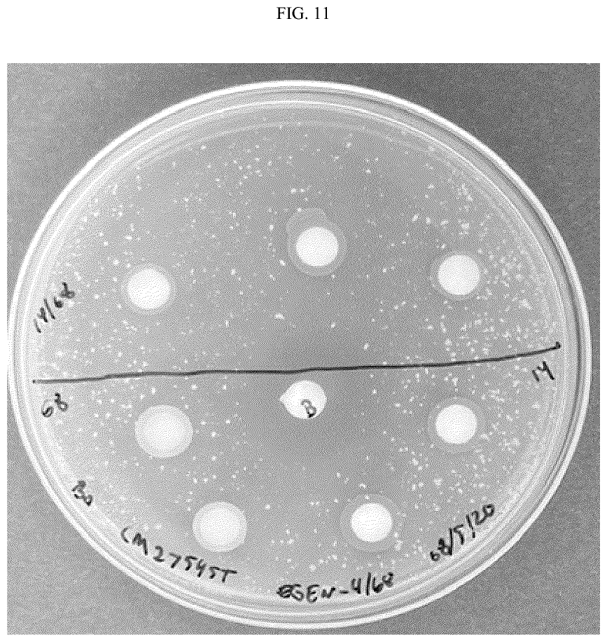

As illustrated in FIG. 11, a lawn of the Corynebacterium matruchotii strain CM2754T was confronted with 50 uL disks of EGEN 14/68 coculture (top), EGEN-68 (bottom left), EGEN-14 (bottom right), and a bleach control.

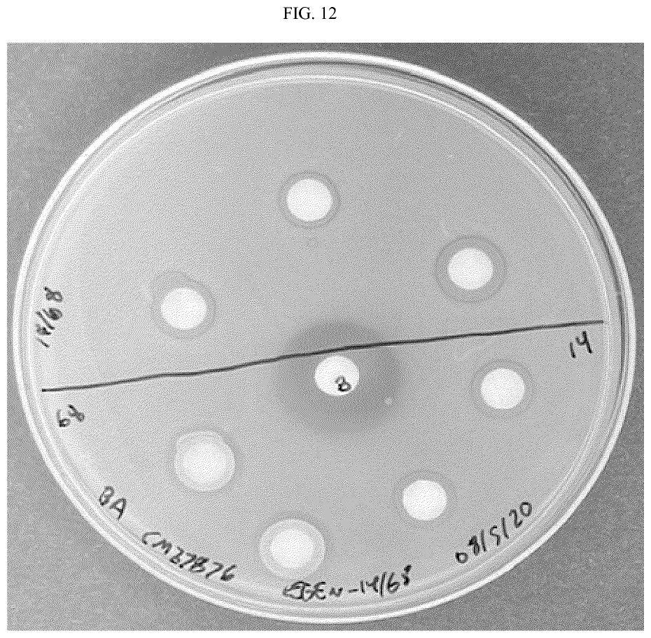

As illustrated in FIG. 12, a lawn of the Corynebacterium matruchotii strain CM37876 was confronted with 50 uL disks of EGEN 14/68 coculture (top), EGEN-68 (bottom left), EGEN-14 (bottom right), and a bleach control.

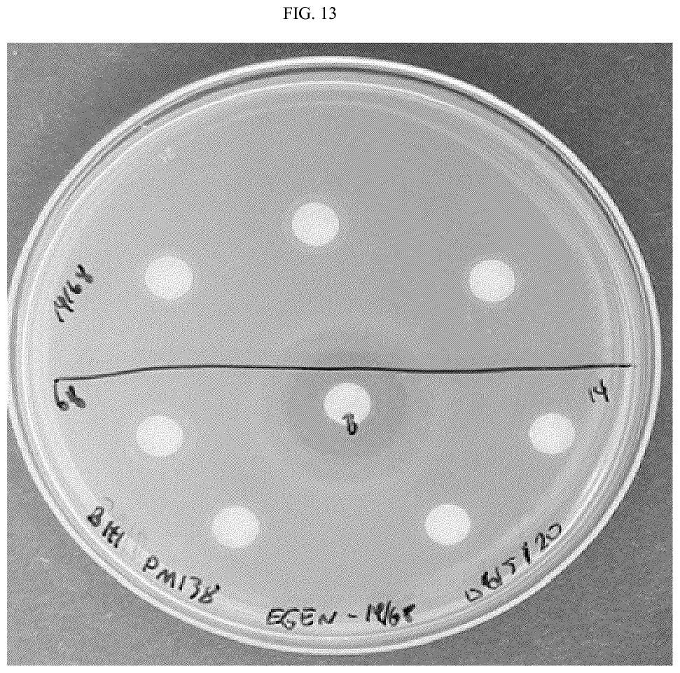

As illustrated in FIG. 13, a lawn of the Proteus mirabilis strain PM138 was confronted with 50 uL disks of EGEN 14/68 coculture (top), EGEN-68 (bottom left), EGEN-14 (bottom right), and a bleach control. None of the challenging agents cleared the lawn except for the bleach control.

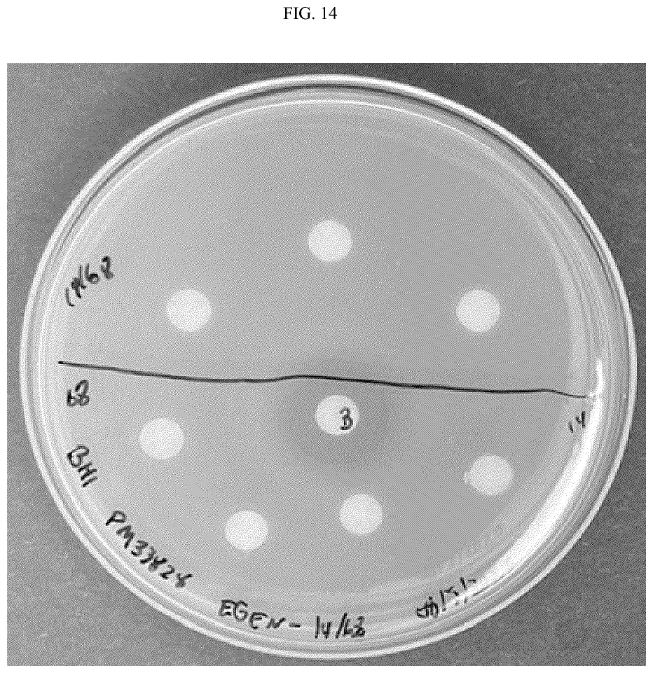

As illustrated in FIG. 14, a lawn of the Proteus mirabilis strain PM33828 was confronted with 50 uL disks of EGEN 14/68 coculture (top), EGEN-68 (bottom left), EGEN-14 (bottom right), and a bleach control. None of the challenging agents cleared the lawn except for the bleach control.

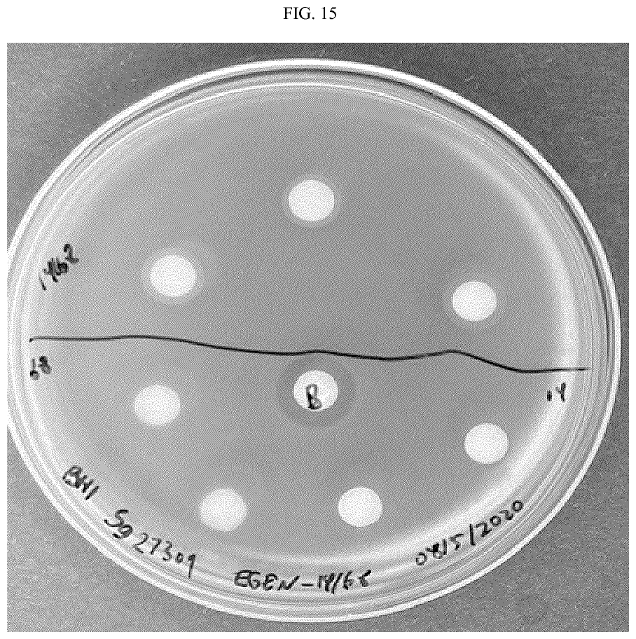

As illustrated in FIG. 15, a lawn of the Streptococcus gordonii strain SG27308 was confronted with 50 uL disks of EGEN 14/68 coculture (top), EGEN-68 (bottom left), EGEN-14 (bottom right), and a bleach control.

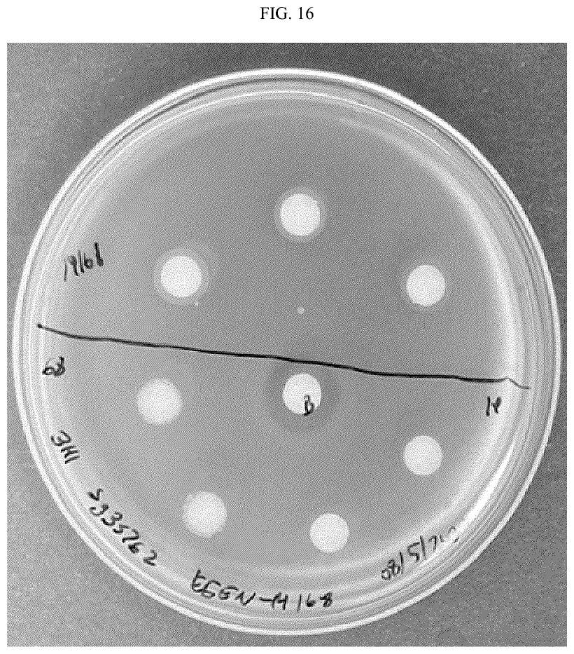

As illustrated in FIG. 16, a lawn of the Streptococcus gordonii strain SG35762 was confronted with 50 uL disks of EGEN 14/68 coculture (top), EGEN-68 (bottom left), EGEN-14 (bottom right), and a bleach control.

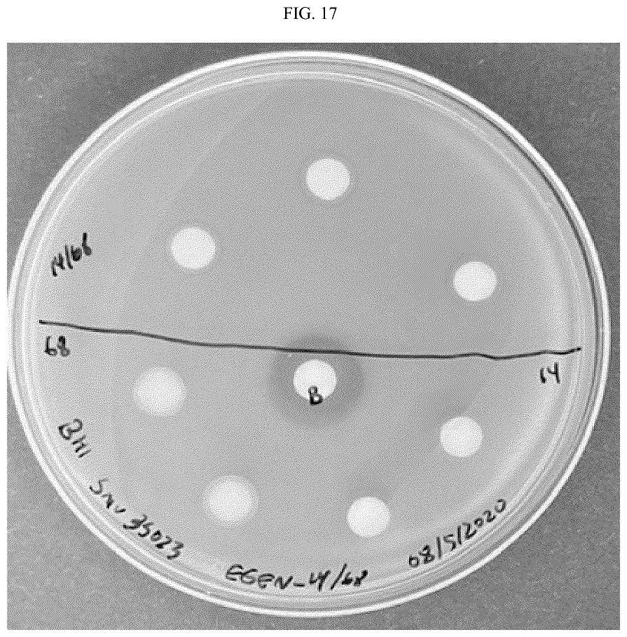

As illustrated in FIG. 17, a lawn of the Streptococcus mutans strain Smu35023 was confronted with 50 uL disks of EGEN 14/68 coculture (top), EGEN-68 (bottom left), EGEN-14 (bottom right), and a bleach control.

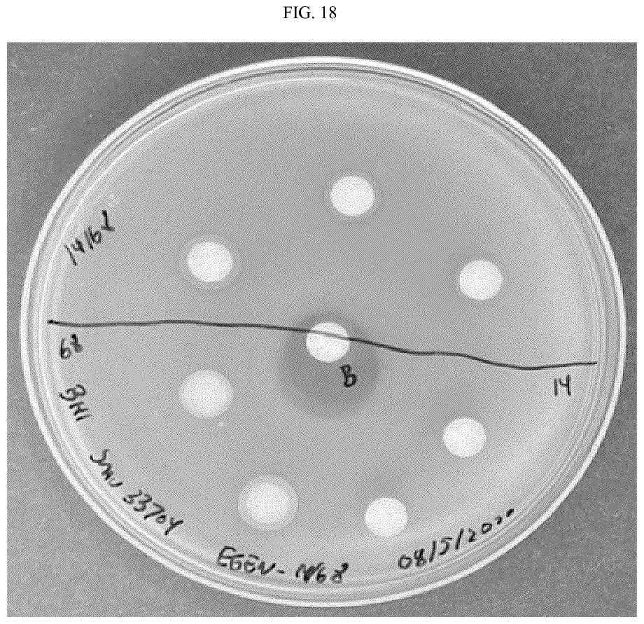

As illustrated in FIG. 18, a lawn of the Streptococcus mutans strain Smu33704 was confronted with 50 uL disks of EGEN 14/68 coculture (top), EGEN-68 (bottom left), EGEN-14 (bottom right), and a bleach control.

A number of embodiments of the invention have been described. Nevertheless, it can be understood that various modifications may be made without departing from the spirit and scope of the invention. Accordingly, other embodiments are within the scope of the following claims.

1. A pharmaceutical composition or a formulation comprising:
(a)
(1) a Streptococcus sanguinis bacterial strain;
(2) a Staphylococcus epidermidis bacterial strain,
wherein the Streptococcus sanguinis and/or the Staphylococcus epidermidis in the product of manufacture, pharmaceutical composition or a formulation are substantially live and viable and are capable of secreting a bacteriocin capable of inhibiting or slowing the growth of a biofilm-forming bacterium or inhibiting or slowing the formation of a biofilm; or
(3) a combination or mixture of bacterial strain comprising at least one bacterial strain from (1) and at least one bacterial strain from (2);
(b) a supernatant or culture medium of a culture or fermentation of the Streptococcus sanguinis and/or Staphylococcus epidermidis bacterial strain, wherein the supernatant or culture medium comprises a bacteriocin capable of inhibiting or slowing the growth of a biofilm-forming bacterium or inhibiting or slowing the formation of a biofilm;
(c) a fraction or isolate of the supernatant or culture medium of (b), wherein the fraction or isolate comprises a bacteriocin capable of inhibiting or slowing the growth of a biofilm-forming bacterium or inhibiting or slowing the formation of a biofilm,
wherein optionally the fraction or isolate is prepared by a column chromatography of the supernatant or culture medium, and the fraction or isolate comprises an eluate fraction of the column capable of inhibiting or slowing the growth of a biofilm-forming bacterium or inhibiting or slowing the formation of a biofilm; or
(d) the product of manufacture, pharmaceutical composition or a formulation of (a), (b) or (c), further comprising a pharmaceutically acceptable excipient, diluent, or carrier.

2. The pharmaceutical composition or formulation of claim 1, wherein the Streptococcus sanguinis and/or Staphylococcus epidermidis bacterial strain, or bacterial strain of (a)(2) or (a)(2), is lyophilized or freeze-dried, and optionally a unit dosage comprises lyophilized or freeze-dried viable Streptococcus sanguinis.

3. The pharmaceutical composition or formulation of claim 1, wherein the Streptococcus sanguinis and/or Staphylococcus epidermidis bacterial strain, or bacterial strain of (a)(2) or (a)(2), is present in an amount that comprises from between about 1×10² to about 1×10¹², or 1×10³ to about 1×10¹¹ CFU/gram (g) of the bacterial strain with respect to a total weight of the pharmaceutical composition, or is present in an amount that comprises from about 1×10² to about 1×10¹², or 1×10³ to about 1×10¹¹ CFU per unit dose.

4. The pharmaceutical composition or formulation of claim 1, formulated as or included or contained in: a liquid, a gel, a capsule, a pill, a tablet, a geltab, a lozenge, a powder, a gum, a sachet, a cachet, an elixir, a hydrogel or a viscosity enhancing agent, a suspension, an emulsion, a liposome or a lipid carrier, a hydrogel, a solution, a toothpaste, a mouthwash, a syrup, a food, a chewing gum, a paste, a candy, a confectionary, an aerosol, a lysosome, a microparticle, a nanoparticle, a microsphere, or a lyophilate or an equivalent thereof.

4. The pharmaceutical composition or formulation of claim 1, further comprising: an additional bacteriocin or biofilm disrupting agent; or, a phage, a stabilizer, an enzyme, a surfactant (optionally a biosurfactant), a hydrogel or viscosity enhancing agent, an antibiotic or a antimicrobial, a complexing agent, a buffer, an emulsifier, a natural product, a flavoring agent, a preservative, a tissue (optionally, gingival or periodontal tissue) penetration enhancer, a pharmaceutically acceptable rate modifying agent, a sustained-release polymer, anti-inflammatory agents, immune-suppressive agents, immune-stimulatory agents, dentinal desensitizers, an odor masking agent, or any combination thereof.

6. A method for treating, ameliorating, preventing or reducing the growth of: a biofilm, optionally a biofilm in an oral environment, optionally a biofilm growing on or adherent to a tooth, a medical device (optionally a bone implant, a pin, a mesh, a stent or an artificial valve), an implant or a prosthetic (optionally an ocular lens), optionally an oral implant or oral prosthetic; or, a microbial colony found in a biofilm, comprising: administering in an individual in need thereof, or applying to a surface in need thereof, a pharmaceutical composition or formulation of claim 1.

7. A method for treating, ameliorating, preventing or reducing the growth of, a biofilm-associated microorganism, or converting the biofilm-associated microorganism to a planktonic state, comprising: administering in an individual in need thereof, or applying to a surface in need thereof, a pharmaceutical composition or formulation of claim 1,
wherein optionally the biofilm-associated microorganism comprises a bacterium from a genus or a species of: Actinomycetes, Bacillus, Listeria (e.g., L. monocytogenes), Staphylococcus, Escherichia (e.g., E. coli), Pseudomonas (e.g., P. aeruginosa), Corynebacterium, Haemophilus, Aggregribacter, Porphyromonas, Neisseria, Capnocytophaga, Fusobacterium and/or Leptotrichia, and/or a lactic acid bacteria (LAB) (e.g., a Bifidobacterium, Lactobacillus, Lactococcus (e.g., L. lactis), Leuconostoc, Pediococcus, Streptococcus (e.g., S. sanguinus), Aerococcus, Alloiococcus, Carnobacterium, Dolosigranulum, Enterococcus, Oenococcus, Tetragenococcus, Vagococcus or Weissella,
and optionally the pharmaceutical composition or formulation is administered to or applied to or on: an oral mucosa or periodontal tissue, a tongue, a gut or a colon, a sinus mucosa, a vaginal mucosa, a stomach, skin, bladder, urethral mucosa, a ureter, an ear, bronchial mucosa, a trachea, a pharynx or a lung.

8. A method for treating, ameliorating, preventing or reducing the severity of or slowing the progress of a periodontitis or a gingivitis, comprising: administering in an individual in need thereof, or applying to a surface in need thereof, a pharmaceutical composition or formulation of claim 1.

9. A method for decreasing breath odor, or for increasing the freshness of breath, comprising: administering in an individual in need thereof, or applying to a surface in need thereof, a pharmaceutical composition or formulation of claim 1.

10. A method for improving the cosmetic appearance of teeth, comprising: administering in an individual in need thereof, or applying to a tooth surface in need thereof, a pharmaceutical composition or formulation of claim 1.

11. A method for restoring a normal oral microbiome in a smoker, a vaper or a tobacco chewer, or for increasing the amount of Streptococcus sanguinis and/or Staphylococcus epidermidis bacterial strain, or bacterial strain of claim 1(a)(2) or 1(a)(2), in an in vivo environment, optionally an oral environment, comprising: administering in an individual in need thereof, a pharmaceutical composition or formulation of claim 1.

12. A method of claim 1, wherein the pharmaceutical composition or formulation is contained in or on (optionally, coated on) or delivered using a delivery device, a syringe, vial, cartridge, an implant, a dressing or patch, a hydrogel, an implantation device, a mouthguard, an orthodontic appliance, a chip or slow release chip, a filament, a needle, a bandage, a gauze, or equivalent thereof.

13. A delivery device, a syringe, vial, cartridge, an implant, a mesh, a fiber, a plug, a tube, a coating, a rod, a dressing or patch, a tray or oral appliance, a hydrogel, a chip or slow release chip, a filament, a needle, a bandage, a gauze, or equivalent thereof, comprising or having stored or carried therein a pharmaceutical composition or formulation of claim 1.

14. (canceled)

15. The pharmaceutical composition or formulation of claim 1, wherein the Streptococcus sanguinis bacterial strain comprises or has contained therein a 16S rRNA gene sequence with at least 96%, 97%, 98%, 99% or 99.5% or more, or complete (100%) percent sequence identity to the sequence of SEQ ID NO: 1 (a strain having complete (100%) percent sequence identity to SEQ ID NO: 1 is also called EGEN14),
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

16. The pharmaceutical composition or formulation of claim 1, wherein the Streptococcus sanguinis bacterial strain comprises or has contained therein a 16S rRNA gene sequence having at least 96%, 97%, 98%, 99% or 99.5% or more, or complete (100%) percent sequence identity to a16S rRNA gene of a Streptococcus sanguinis SK36 strain),
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

17. The pharmaceutical composition or formulation of claim 1, wherein the Staphylococcus epidermidis bacterial strain comprises or has contained therein a 16S rRNA gene sequence with at least 96%, 97%, 98%, 99% or 99.5% or more, or complete (100%) percent sequence identity to the sequence of SEQ ID NO:2 (a strain having complete (100%) percent sequence identity to SEQ ID NO:2 is also called EGEN68),
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

18. The pharmaceutical composition or formulation of claim 1, wherein the Staphylococcus epidermidis bacterial strain is a strain having ATCC deposit no. 12228.

19. The pharmaceutical composition or formulation of claim 1, wherein the Staphylococcus epidermidis bacterial strain comprises or has contained therein a 16S rRNA gene sequence having at least 96%, 97%, 98%, 99% or 99.5% or more, or complete (100%) percent sequence identity to a16S rRNA gene of a Staphylococcus epidermidis strain ATCC deposit no. 12228,
wherein optionally the sequence identity is determined by the Smith-Waterman homology search algorithm using: a linear gap with score scheme of 1 for match and −2 for mismatch; or, a gap search with a gap open penalty of 12 and a gap extension penalty of 2, and a BLOSUM matrix of 62.

20. The pharmaceutical composition or formulation of claim 1, wherein the supernatant or culture medium is free or substantially free of any bacteria, and optionally a culture or a fermentation supernatant or culture medium is filtered to remove substantially all or all bacteria from the supernatant or culture medium.

21. The pharmaceutical composition or formulation of claim 1, wherein the biofilm-forming bacterium is a genus or a species of Actinomycetes, Bacillus, Listeria (optionally, a L. monocytogenes), Staphylococcus, Escherichia (optionally, a E. coli), Pseudomonas (optionally, a P. Aeruginosa), Corynebacterium, Haemophilus, Aggregribacter, Porphyromonas, Neisseria, Capnocytophaga, Fusobacterium and/or Leptotrichia, and/or a lactic acid bacteria (LAB) (optionally, a Bifidobacterium, Lactobacillus, Lactococcus (optionally, a L. lactis), Leuconostoc, Pediococcus, Streptococcus (optionally, S. sanguinus), Aerococcus, Alloiococcus, Carnobacterium, Dolosigranulum, Enterococcus, Oenococcus, Tetragenococcus, Vagococcus or Weissella.


SEQUENCE LISTING (2 sequences)

SEQ ID NO: 1 - DNA, 2210 bp, Streptococcus sanguinis
ggtataggtgatatactaatatagttgtcgcgaaagacaaagccctttgaaaactgaacaagacgaaccaagtgcagggtgacatagagatatgtaacctgtcaaaaaaacgaaaataaatctgtcagtggacagtaatgagtgcgaactcaaactttttaatgagagtttgatcctggctcaggacgaacgctggcggcgtgcctaatacatgcaagtagaacgctgaagagaggagcttgctcttcttggatgagttgcgaacgggtgagtaacgcgtaggtaacctgcctggtagcgggggataactattggaaacgatagctaataccgcataaaattgattattgcatgataattaattgaaagatgcaactgcatcactaccagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgtaagagaagaacgggtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtatgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttctaaggagtccgtaaggacacacggaatgcacttgggtcttgtttagttttgagagggctatgtggggccttagctcagctgggagagcgcctgctttgcacgcaggaggtcagcggttcgatcccgctaggctccattatctagtttgactagataaagaacttgtccattgaaaattgaataccgatatcaaatagtaacaagaaaataaaccgaaaacgctgtgaatattaatgagtttaagactgaaaggtcaaaaataaggttaagttagtaagggcgcacggtggatgccttggcactaggagccgatgaaggacgtgacaaacgacgaaatgcctcggggagctgtaagtaagcttcgatccgggggtgtccgaatgggggaacccaacaggttgatgcctgttacccatttctgttaagggaatgaggaggaagacgcagtgaactgaaacatctaagtagctgcaggaagagaaagcaaaagcgattgccttagta

SEQ ID NO: 2 - DNA, 1539 bp, Staphylococcus epidermidis
agagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacct